(19) United States
(12) Patent Application Publication
(10) Pub. No.: US 2025/0050046 A1
(43) Pub. Date: Feb. 13, 2025

(54) PATIENT INTERFACE SYSTEM

(71) Applicant: ResMed Pty Ltd, Bella Vista (AU)

(72) Inventors: Balint FRANKO, Sydney (AU); Jing CHEN, Singapore (SG); Abdul Rashid Bin BASIRON, Singapore (SG)

(21) Appl. No.: 18/797,344

(22) Filed: Aug. 7, 2024

(30) Foreign Application Priority Data
Aug. 10, 2023 (AU) 2023902537

(51) Int. Cl. A61M 16/06

(52) U.S. Cl. CPC A61M 16/0622; A61M 16/0633; A61M 2202/0007; A61M 2202/02; A61M 2205/0216; A61M 2205/3331; A61M 2209/088; A61M 2210/0618

(57) ABSTRACT

The present technology relates to a patient interface for use in delivering breathable gas to a patient. The patient interface may comprise a cushion module and a frame. The cushion module may comprise a cushion and a cushion clip. The cushion clip may be configured to hold the cushion module in a substantially fixed position relative to the frame in use. In some forms, the frame and the cushion module are configured to connect together interlocks between the cushion module and the frame is configured to flex to alter a distance between interlocks, and wherein flexing the frame enables the cushion module to be disconnected from the frame.

### 1 CROSS-REFERENCE TO RELATED APPLICATIONS

This application claims the benefit of Australian Patent Application No. 2023902537, filed Aug. 10, 2023, which is incorporated herein by reference in its entirety.

### 2 BACKGROUND OF THE TECHNOLOGY

### 2.1 Field of the Technology

The present technology relates to one or more of the screening, diagnosis, monitoring, treatment, prevention and amelioration of respiratory-related disorders. The present technology also relates to medical devices or apparatus, and their use.

In particular, the present technology relates to patient interfaces for use in respiratory therapy systems for delivering respiratory therapy to patients. Still more particularly, the present technology relates to patient interfaces that may be readily disassembled and/or reassembled.

### 2.2 Description of the Related Art

### 2.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD) and Chest wall disorders.

Obstructive Sleep Apnea (OSA), a form of Sleep Disordered Breathing (SDB), is characterised by events including occlusion or obstruction of the upper air passage during sleep. It results from a combination of an abnormally small upper airway and the normal loss of muscle tone in the region of the tongue, soft palate and posterior oropharyngeal wall during sleep. The condition causes the affected patient to stop breathing for periods typically of 30 to 120 seconds in duration, sometimes 200 to 300 times per night. It often causes excessive daytime somnolence, and it may cause cardiovascular disease and brain damage. The syndrome is a common disorder, particularly in middle aged overweight males, although a person affected may have no awareness of the problem. See U.S. Pat. No. 4,944,310 (Sullivan).

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterised by repetitive de-oxygenation and re-oxygenation of the arterial blood. It is possible that CSR is harmful because of the repetitive hypoxia. In some patients CSR is associated with repetitive arousal from sleep, which causes severe sleep disruption, increased sympathetic activity, and increased afterload. See U.S. Pat. No. 6,532,959 (Berthon-Jones).

Respiratory failure is an umbrella term for respiratory disorders in which the lungs are unable to inspire sufficient oxygen or exhale sufficient CO2 to meet the patient's needs. Respiratory failure may encompass some or all of the following disorders.

A patient with respiratory insufficiency (a form of respiratory failure) may experience abnormal shortness of breath on exercise.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive: (i) Rapidly progressive disorders: Characterised by muscle impairment that worsens over months and results in death within a few years (e.g. Amyotrophic lateral sclerosis (ALS) and Duchenne muscular dystrophy (DMD) in teenagers); (ii) Variable or slowly progressive disorders: Characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

A range of therapies have been used to treat or ameliorate such conditions. Furthermore, otherwise healthy individuals may take advantage of such therapies to prevent respiratory disorders from arising. However, these have a number of shortcomings.

### 2.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 2.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube or endotracheal tube. In some forms, the comfort and effectiveness of these therapies may be improved.

### 2.2.2.2 Flow Therapies

Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure.

Another form of flow therapy is long-term oxygen therapy (LTOT) or supplemental oxygen therapy. Doctors may prescribe a continuous flow of oxygen enriched air at a specified oxygen concentration (from 21%, the oxygen fraction in ambient air, to 100%) at a specified flow rate (e.g., 1 litre per minute (LPM), 2 LPM, 3 LPM, etc.) to be delivered to the patient's airway.

### 2.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

### 2.2.3.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the patient interface may not include a seal sufficient to facilitate delivery to the airways of a supply of gas at a positive pressure of about 10 cmH₂O.

Certain other mask systems may be functionally unsuitable for the present field. For example, purely ornamental masks may be unable to maintain a suitable pressure. Mask systems used for underwater swimming or diving may be configured to guard against ingress of water from an external higher pressure, but not to maintain air internally at a higher pressure than ambient.

Certain masks may be clinically unfavourable for the present technology e.g. if they block airflow via the nose and only allow it via the mouth.

Certain masks may be uncomfortable or impractical for the present technology if they require a patient to insert a portion of a mask structure in their mouth to create and maintain a seal via their lips.

Certain masks may be impractical for use while sleeping, e.g. for sleeping while lying on one's side in bed with a head on a pillow.

The design of a patient interface presents a number of challenges. The face has a complex three-dimensional shape. The size and shape of noses and heads varies considerably between individuals. Since the head includes bone, cartilage and soft tissue, different regions of the face respond differently to mechanical forces. The jaw or mandible may move relative to other bones of the skull. The whole head may move during the course of a period of respiratory therapy.

As a consequence of these challenges, some masks suffer from being one or more of obtrusive, aesthetically undesirable, costly, poorly fitting, difficult to use, and uncomfortable especially when worn for long periods of time or when a patient is unfamiliar with a system. Wrongly sized masks can give rise to reduced compliance, reduced comfort and poorer patient outcomes. Masks designed solely for aviators, masks designed as part of personal protection equipment (e.g. filter masks), SCUBA masks, or for the administration of anaesthetics may be tolerable for their original application, but nevertheless such masks may be undesirably uncomfortable to be worn for extended periods of time, e.g., several hours. This discomfort may lead to a reduction in patient compliance with therapy. This is even more so if the mask is to be worn during sleep.

CPAP therapy is highly effective to treat certain respiratory disorders, provided patients comply with therapy. If a mask is uncomfortable, or difficult to use a patient may not comply with therapy. Since it is often recommended that a patient regularly wash their mask, if a mask is difficult to clean (e.g., difficult to assemble or disassemble), patients may not clean their mask and this may impact on patient compliance. Feedback from some patients has shown that a patient may be more likely to comply with therapy if a patient interface is designed in a way that it does not look overly ‘medical’ in appearance.

While a mask for other applications (e.g. aviators) may not be suitable for use in treating sleep disordered breathing, a mask designed for use in treating sleep disordered breathing may be suitable for other applications.

For these reasons, patient interfaces for delivery of CPAP during sleep form a distinct field.

Patient interfaces for CPAP therapy are typically comprised of a number of components assembled together. Patients may need or desire to be able to disassemble some of the components, for example to clean the components, or to replace them when they are damaged, and then to re-assemble the patient interface. Some patient interfaces can be difficult to disassemble and/or re-assemble, particularly for patients with impaired dexterity, vision or by a person with limited experience in using this type of medical device. It is desirable for patient interfaces to be easily able to be disassembled and/or easily able to be re-assembled.

Patient interfaces typically have a limited lifespan since they become damaged after a while, or unable to form a desired level of seal with the patient during use. If a patient interface is disposed of, it is desirable to be able to recycle it. Typically, the components forming patient interfaces are formed from a variety of materials, some of which may be easily recyclable and some of which may not be. In addition, the recycling process may be different for different components, for example the components may need to be sorted separately. If a component is formed from a plurality of sub-components formed of different materials, some of which are easily recyclable and some of which are not, the whole component may be difficult to recycle.

### 2.2.3.1.1 Seal-Forming Structure

Patient interfaces may include a seal-forming structure. Since it is in direct contact with the patient's face, the shape and configuration of the seal-forming structure can have a direct impact the effectiveness and comfort of the patient interface.

A patient interface may be partly characterised according to the design intent of where the seal-forming structure is to engage with the face in use. In one form of patient interface, a seal-forming structure may comprise a single element that surrounds both nares in use. Such single element may be designed to, for example, overlay an upper lip region and a nasal bridge region of a face. In one form of patient interface a seal-forming structure may comprise an element that surrounds a mouth region in use, e.g. by forming a seal on a lower lip region of a face. In one form of patient interface, a seal-forming structure may comprise a single element that surrounds both nares and a mouth region in use. These different types of patient interfaces may be known by a variety of names by their manufacturer including nasal masks, full-face masks, nasal pillows, nasal puffs and oro-nasal masks.

A seal-forming structure that may be effective in one region of a patient's face may be inappropriate in another region, e.g. because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design fit and be comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface, and is intended to seal against the patient's face when force is applied to the patient interface with the seal-forming structure in confronting engagement with the patient's face. The seal-forming structure may include an air or fluid filled cushion, or a moulded or formed surface of a resilient seal element made of an elastomer such as a rubber. With this type of seal-forming structure, if the fit is not adequate, there will be gaps between the seal-forming structure and the face, and additional force will be required to force the patient interface against the face in order to achieve a seal.

Another type of seal-forming structure incorporates a flap seal of thin material positioned about the periphery of the mask so as to provide a self-sealing action against the face of the patient when positive pressure is applied within the mask. Like the previous style of seal forming portion, if the match between the face and the mask is not good, additional force may be required to achieve a seal, or the mask may leak. Furthermore, if the shape of the seal-forming structure does not match that of the patient, it may crease or buckle in use, giving rise to leaks.

Another type of seal-forming structure may comprise a friction-fit element, e.g. for insertion into a naris, however some patients find these uncomfortable.

Another form of seal-forming structure may use adhesive to achieve a seal. Some patients may find it inconvenient to constantly apply and remove an adhesive to their face.

A range of patient interface seal-forming structure technologies are disclosed in the following patent applications, assigned to ResMed Limited: WO 1998/004,310; WO 2006/074,513; WO 2010/135,785.

There is a need for a seal-forming structure to provide an effective seal when used by patients having facial features with a range of different shapes and sizes while being low cost and easy to manufacture, and easy and comfortable for a patient to use.

### 2.2.3.1.2 Positioning and Stabilising

A seal-forming structure of a patient interface used for positive air pressure therapy is subject to the corresponding force of the air pressure to disrupt a seal. Thus a variety of techniques have been used to position the seal-forming structure, and to maintain it in sealing relation with the appropriate portion of the face.

One technique is the use of adhesives. See for example US Patent Application Publication No. US 2010/0000534. However, the use of adhesives may be uncomfortable for some.

Another technique is the use of one or more straps and/or stabilising harnesses. Many such harnesses suffer from being one or more of ill-fitting, bulky, uncomfortable and awkward to use. There is a need for a positioning and stabilising structure that is easy and intuitive for a patient to use.

### 2.2.3.1.3 Pressurised Air Conduit

In one type of treatment system, a flow of pressurised air is provided to a patient interface through a conduit in an air circuit that fluidly connects to the patient interface at a location that is in front of the patient's face when the patient interface is positioned on the patient's face during use. The conduit may extend from the patient interface forwards away from the patient's face.

2.2.3.1.4 Pressurised Air Conduit used for Positioning/Stabilising the Seal-Forming Structure

Another type of treatment system comprises a patient interface in which a tube that delivers pressurised air to the patient's airways also functions as part of the headgear to position and stabilise the seal-forming portion of the patient interface at the appropriate part of the patient's face. This type of patient interface may be referred to as having “conduit headgear” or “headgear tubing”. Such patient interfaces allow the conduit in the air circuit providing the flow of pressurised air from a respiratory pressure therapy (RPT) device to connect to the patient interface in a position other than in front of the patient's face. One example of such a treatment system is disclosed in US Patent Publication No. US 2007/0246043, the contents of which are incorporated herein by reference, in which the conduit connects to a tube in the patient interface through a port positioned in use on top of the patient's head.

It is desirable for patient interfaces incorporating headgear tubing to be comfortable for a patient to wear over a prolonged duration when the patient is asleep, form an air-tight and stable seal with the patient's face, while also able to fit a range of patient head shapes and sizes.

### 2.2.3.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

### 2.2.3.3 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

### 2.2.3.4 Humidifier

Delivery of a flow of air without humidification may cause drying of airways. The use of a humidifier with an RPT device and the patient interface produces humidified gas that minimizes drying of the nasal mucosa and increases patient airway comfort. In addition, in cooler climates, warm air applied generally to the face area in and about the patient interface is more comfortable than cold air.

### 2.2.3.5 Vent Technologies

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

The vent may comprise an orifice and gas may flow through the orifice in use of the mask. Many such vents are noisy. Others may become blocked in use and thus provide insufficient washout. Some vents may be disruptive of the sleep of a bed partner 1100 of the patient 1000, e.g. through noise or focused airflow.

ResMed Limited has developed a number of improved mask vent technologies. See International Patent Application Publication No. WO 1998/034,665; International Patent Application Publication No. WO 2000/078,381; U.S. Pat. No. 6,581,594; US Patent Application Publication No. US 2009/0050156; US Patent Application Publication No. 2009/0044808.

### 3 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

An aspect of certain forms of the present technology is to provide apparatus that improve the compliance of patients with respiratory therapy.

One aspect of the present technology comprises a patient interface for use in delivering breathable gas to a patient.

An aspect of the present technology is a patient interface that is easy to disassemble and/or re-assemble. In particular, the patient interface may be easy to disassemble and/or re-assemble by a person who does not have medical training, by a person who has limited dexterity, vision or by a person with limited experience in using this type of medical device.

Another aspect of the present technology is a patient interface that is able to be disassembled into separate components in a manner that aids recycling some of the components.

An aspect of the present technology relates to a patient interface for use in delivering breathable gas to a patient. The patient interface may comprise a cushion module comprising a cushion and a cushion clip. The patient interface may further comprise a frame. The cushion, cushion clip and frame may be configured to be readily disassembled from each other and re-assembled by a user.

An aspect of the present technology relates to cushion module for a patient interface for use in delivering breathable gas to a patient. The cushion module may comprise a cushion and a cushion clip. The cushion and cushion clip may be configured to be readily disassembled from each other and re-assembled by a user. The first cushion clip portion may comprise one or more first magnetic members and the second cushion clip portion may comprise one or more second magnetic members.

An aspect of the present technology relates to a patient interface for use in delivering breathable gas to a patient. The patient interface may comprise a cushion module and a frame. The frame and the cushion module may be configured to connect together via a first interlock between a first cushion module interlock portion on a first lateral side of a non-patient-facing side of the cushion module and a first frame interlock portion on a first lateral side of a patient-facing side of the frame, and via a second interlock between a second cushion module interlock portion on a second lateral side of a non-patient-facing side of the cushion module and a second frame interlock portion on a second lateral side of a patient-facing side of the frame. The frame may be configured to flex to alter a distance between the first frame interlock portion and the second frame interlock portion. Flexing the frame to increase the distance between the first frame interlock portion and the second frame interlock portion may enable the cushion module to be disconnected from the frame.

An aspect of the present technology relates to a patient interface for use in delivering breathable gas to a patient. The patient interface may comprise a cushion module. The patient interface may further comprise a frame. The cushion module may form a plenum chamber substantially defining a volume of space and pressurisable to a therapeutic pressure of at least 4 cmH2O above ambient air pressure. The plenum chamber may comprise an anterior wall substantially forming an anterior boundary of the volume of space during use. The plenum chamber may form a plenum chamber inlet port configured to receive a flow of breathable gas at the therapeutic pressure for breathing by the patient. The cushion module may form a seal-forming structure provided to the plenum chamber. The seal-forming structure may be configured to form a seal with a region of the patient's face surrounding an entrance to the patient's airways. The seal-forming structure may have an opening therein such that in use the flow of breathable gas is delivered to the entrance to the patient's airways. The seal-forming structure may be configured to maintain said therapeutic pressure in the plenum chamber throughout the patient's respiratory cycle in use. The frame may comprise a frame body forming a frame opening allowing the flow of breathable gas to be delivered to the plenum chamber. The frame may further comprise two or more headgear connectors for connecting with headgear. The cushion module may comprise a cushion and a cushion clip. The cushion clip may be configured to hold the cushion module in a substantially fixed position relative to the frame in use. The cushion, cushion clip and frame may be configured to be readily disassembled from each other and re-assembled by a user.

In certain forms, the cushion clip may be formed from a material that is readily recyclable.

In certain forms, the cushion clip may comprise a first cushion clip portion and a second cushion clip portion. The first cushion clip portion and the second cushion clip portion may be configured to connect together such that, in an assembled configuration of the patient interface, a portion of the cushion is sandwiched between the first cushion clip portion and the second cushion clip portion, and the second cushion clip portion connects to the frame. The first cushion clip portion and the second cushion clip portion may be configured so that, in a disassembled configuration of the patient interface, the first cushion clip portion and the second clip portion are at least partially separated so that the cushion may be readily disassembled from the cushion clip by a user.

In certain forms, the cushion clip may be configured so that the first cushion clip portion is completely disconnected from the second cushion clip portion in the disassembled configuration. In other forms, the cushion clip may be configured so that the first cushion clip portion is partly connected to the second cushion clip portion in the disassembled configuration. For example, the first cushion clip portion may be connected to the second cushion clip portion via a hinge.

In certain forms, in the assembled configuration, the first cushion clip portion is magnetically connected to the second cushion clip portion. In certain forms, the first cushion clip portion may comprise one or more first magnetic members and the second cushion clip portion may comprise one or more second magnetic members. In certain forms, the first magnetic members may be configured to be readily removed from the first cushion clip portion by a user and the second magnetic members may be configured to be readily removed from the second cushion clip portion by a user.

In certain forms, the cushion clip may form at least part of the anterior wall. In certain forms, the second cushion clip portion may form at least part of the anterior wall.

In certain forms, the plenum chamber inlet port may be formed in a central region of the anterior wall. In other forms, the plenum chamber inlet port may be a first plenum chamber inlet port located on a first lateral side of the plenum chamber and the plenum chamber may further form a second plenum chamber inlet port located on a second lateral side of the plenum chamber.

In certain forms, the patient interface further may comprise a vent configured to allow a continuous flow of gases exhaled by the patient from an interior of the plenum chamber to ambient. Said vent may be configured to maintain the therapeutic pressure in the plenum chamber in use.

In certain forms, the patient interface may further comprise headgear for positioning and stabilising the patient interface in a therapeutically effective position on the patient's head in use.

An aspect of the present technology relates to a cushion module for a patient interface for use in delivering breathable gas to a patient. The cushion module may form a plenum chamber substantially defining a volume of space and pressurisable to a therapeutic pressure of at least 4 cmH2O above ambient air pressure. The plenum chamber may form a plenum chamber inlet port configured to receive a flow of breathable gas at the therapeutic pressure for breathing by the patient. The cushion module may form a seal-forming structure provided to the plenum chamber. The seal-forming structure may be configured to form a seal with a region of the patient's face surrounding an entrance to the patient's airways. The seal-forming structure may have an opening therein such that in use the flow of breathable gas is delivered to the entrance to the patient's airways. The seal-forming structure may be configured to maintain said therapeutic pressure in the plenum chamber throughout the patient's respiratory cycle in use. The cushion module may comprise a cushion and a cushion clip configured to be readily disassembled from each other and re-assembled by a user. The cushion clip may comprise a first cushion clip portion and a second cushion clip portion. The first cushion clip portion and the second cushion clip portion may be configured to connect together such that, in an assembled configuration of the patient interface, a portion of the cushion is sandwiched between the first cushion clip portion and the second cushion clip portion, and, in a disassembled configuration of the patient interface, the first cushion clip portion and the second clip portion are at least partially separated so that the cushion may be disassembled from the cushion clip. The second cushion clip portion may be configured to connect to a frame of the patient interface. The first cushion clip portion may comprise one or more first magnetic members and the second cushion clip portion may comprise one or more second magnetic members.

An aspect of the present technology relates to a patient interface for use in delivering breathable gas to a patient. The patient interface may comprise a cushion module according to another aspect of the technology. The patient interface may further comprise a frame, The frame may comprise a frame body forming a frame opening allowing the flow of breathable gas to be delivered to the plenum chamber. The frame may further comprise two or more headgear connectors for connecting with headgear. The cushion module and frame may be configured to be readily disassembled from each other and re-assembled by a user.

An aspect of the present technology relates to a patient interface for use in delivering breathable gas to a patient. The patient interface may comprise a cushion module. The patient interface may further comprise a frame. The cushion module may comprise a plenum chamber pressurisable to a therapeutic pressure of at least 4 cmH2O above ambient air pressure, said plenum chamber forming a plenum chamber inlet port configured to receive a flow of breathable gas at the therapeutic pressure for breathing by the patient. The cushion module may further comprise a seal-forming structure provided to the plenum chamber. The seal-forming structure may be configured to form a seal with a region of the patient's face surrounding an entrance to the patient's airways. The seal-forming structure may have an opening therein such that in use the flow of breathable gas is delivered to the entrance to the patient's airways. The seal-forming structure may be configured to maintain said therapeutic pressure in the plenum chamber throughout the patient's respiratory cycle in use. The cushion module may further comprise a first cushion module interlock portion on a first lateral side of a non-patient-facing side of the cushion module. The cushion module may further comprise a second cushion module interlock portion on a second lateral side of the non-patient-facing side of the cushion module. The frame may comprise a frame body forming a frame opening allowing the flow of breathable gas to be delivered to the plenum chamber. The frame may further comprise two or more headgear connectors for connecting with headgear, the headgear connectors being positioned on respective lateral sides of the frame body in use. The frame may further comprise a first frame interlock portion on a first lateral side of a patient-facing side of the frame body. The frame may further comprise a second frame interlock portion on a second lateral side of the patient-facing side of the frame body. The frame and the cushion module may be configured to connect together via a first interlock between the first cushion module interlock portion and the first frame interlock portion and via a second interlock between the second cushion module interlock portion and the second frame interlock portion. The frame may be configured to flex to alter a distance between the first frame interlock portion and the second frame interlock portion. Flexing the frame to increase the distance between the first frame interlock portion and the second frame interlock portion may enable the cushion module to be disconnected from the frame.

In certain forms, the first cushion interlock portion may comprise a first recess, the second cushion interlock portion may comprise a second recess, the first frame interlock portion may comprise a first protrusion, and the second frame interlock portion may comprise a second protrusion.

In certain forms, the patient-facing side of the frame body may be concave.

In certain forms, the first frame interlock portion and the second frame interlock portion may extend outwardly from a patient-facing side of the frame in a direction substantially parallel to a frontal plane of the patient during use.

In certain forms, the cushion module may comprise a cushion and a cushion clip. The first cushion module interlock portion and the second cushion module interlock portion may be provided on the cushion clip.

In certain forms, the cushion clip may form a loop around the plenum chamber inlet port.

In certain forms, when the cushion module is connected to the frame, the cushion clip may form a loop around the frame opening.

In certain forms, the cushion clip may be more rigid than the frame.

In certain forms, the plenum chamber may substantially define a volume of space and the plenum chamber may comprise an anterior wall substantially forming an anterior boundary of the volume of space during use.

In certain forms, the plenum chamber and the frame may together substantially define a volume of space and the frame may form an anterior boundary of the volume of space during use.

In certain forms, the patient interface may further comprise a vent configured to allow a continuous flow of gases exhaled by the patient from an interior of the plenum chamber to ambient. The vent may be configured to maintain the therapeutic pressure in the plenum chamber in use.

In certain forms, the patient interface may further comprise headgear for positioning and stabilising the patient interface in a therapeutically effective position on the patient's head in use.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 4 BRIEF DESCRIPTION OF THE DRAWINGS

The present technology is illustrated by way of example, and not by way of limitation, in the figures of the accompanying drawings, in which like reference numerals refer to similar elements including:

### 4.1 Respiratory Therapy Systems

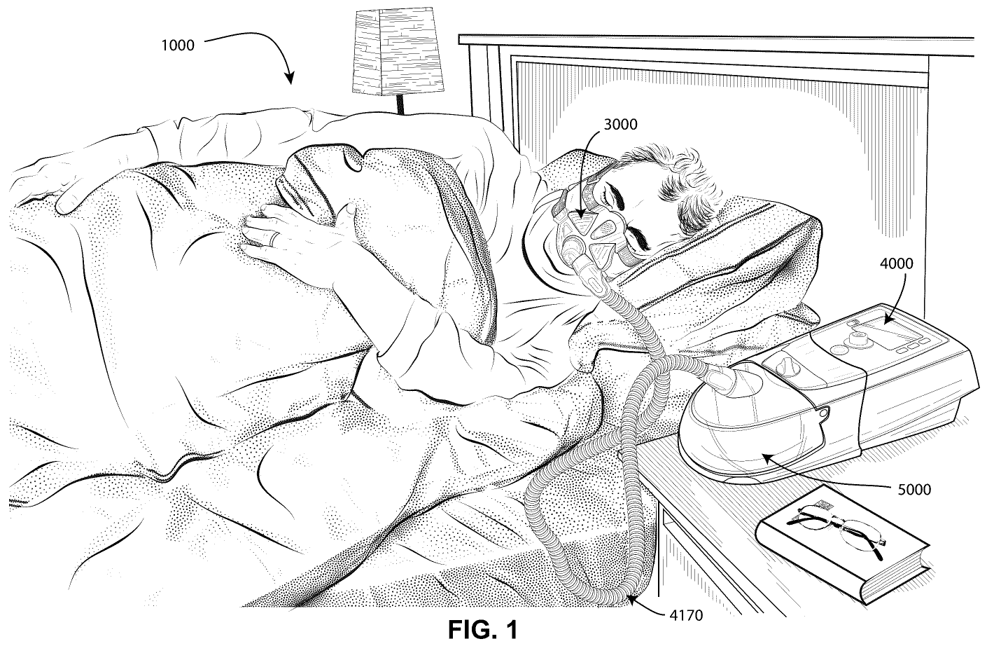

FIG. 1 shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 4.2 Respiratory System and Facial Anatomy

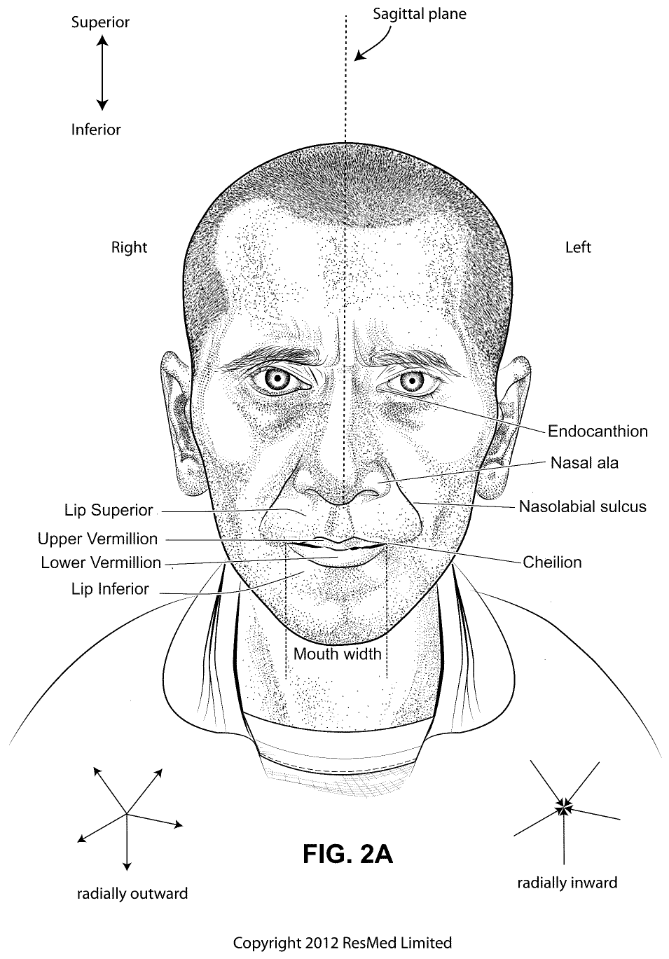

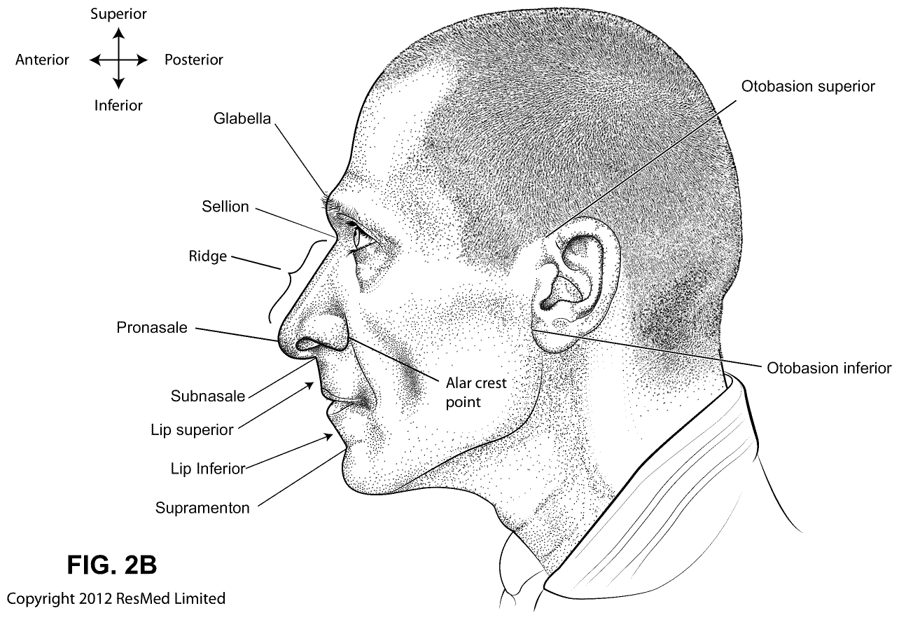

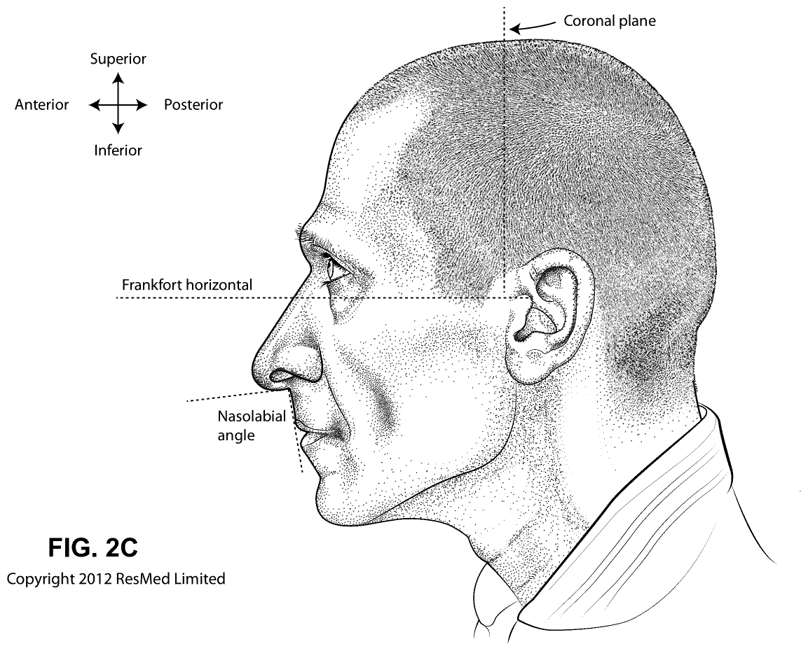

FIG. 2A is a front view of a face with several features of surface anatomy identified including the lip superior, upper vermilion, lower vermilion, lip inferior, mouth width, endocanthion, a nasal ala, nasolabial sulcus and cheilion. Also indicated are the directions superior, inferior, radially inward and radially outward.

FIG. 2B is a side view of a head with several features of surface anatomy identified including glabella, sellion, pronasale, subnasale, lip superior, lip inferior, supramenton, nasal ridge, alar crest point, otobasion superior and otobasion inferior. Also indicated are the directions superior & inferior, and anterior & posterior.

FIG. 2C is a further side view of a head. The approximate locations of the Frankfort horizontal and nasolabial angle are indicated. The coronal plane is also indicated.

### 4.3 Patient Interface

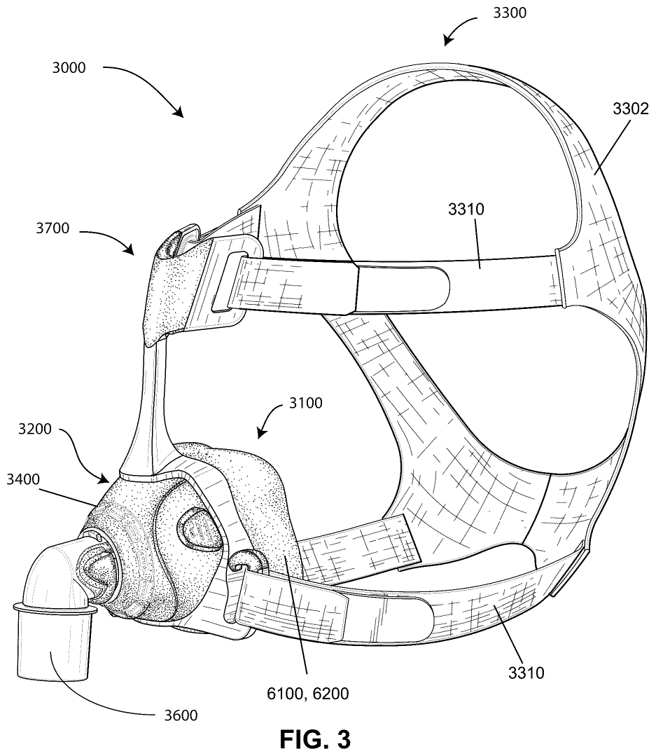

FIG. 3 shows a patient interface in the form of a nasal mask in accordance with one form of the present technology.

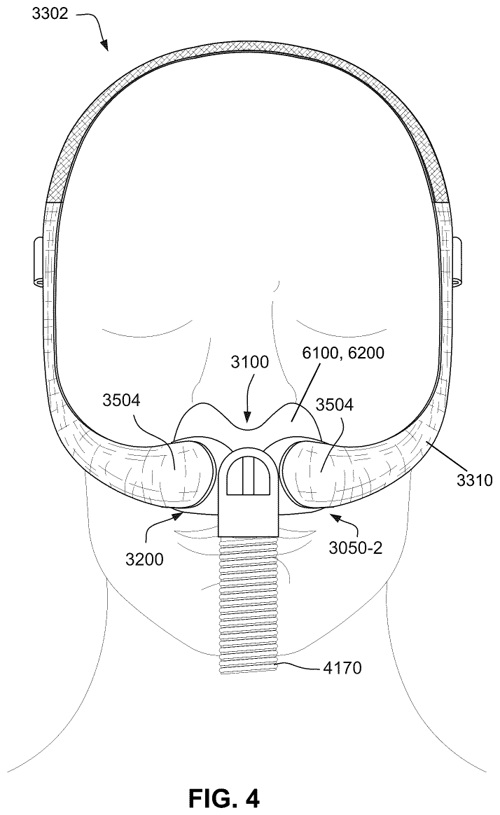

FIG. 4 shows a patient interface in the form of nasal cradle mask in accordance with one form of the present technology.

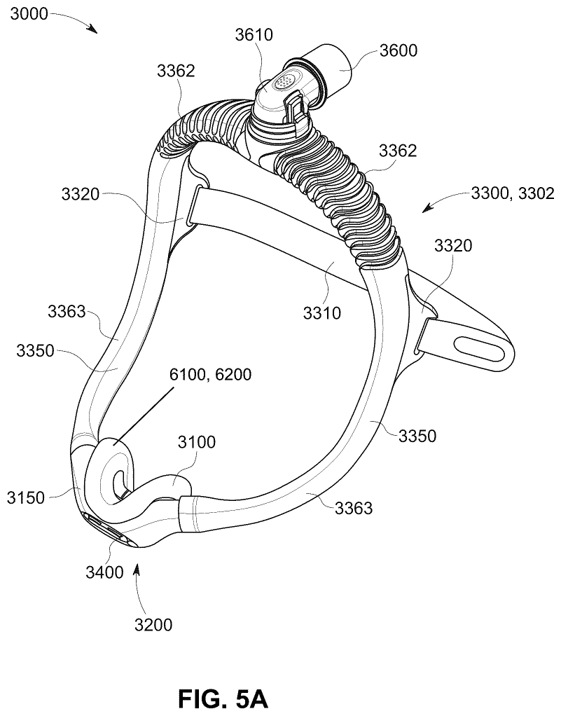

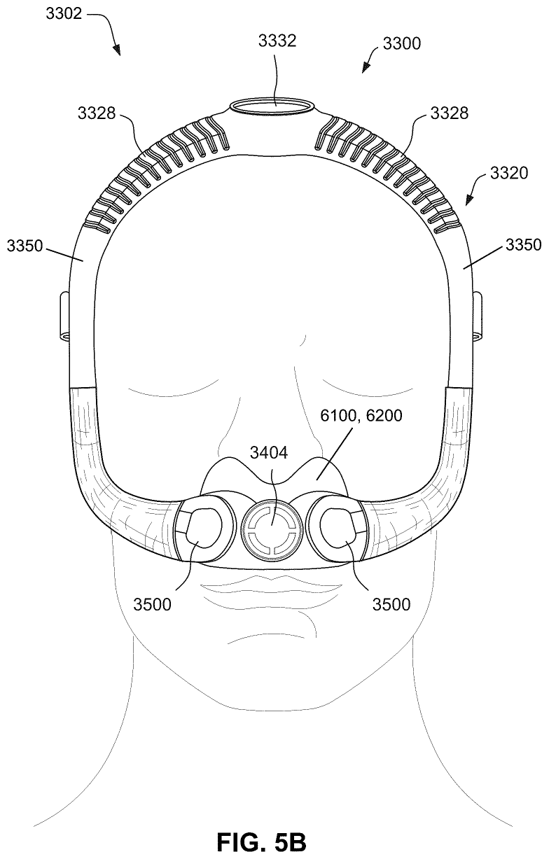

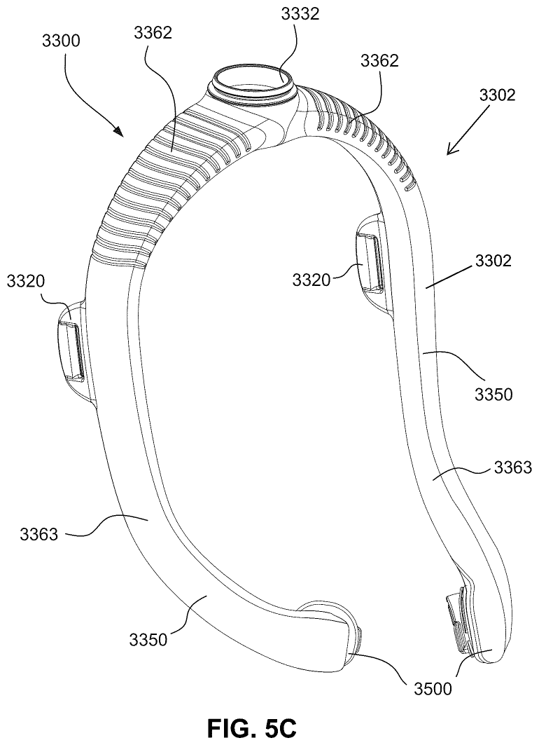

FIG. 5A shows a patient interface in the form of a nasal cradle mask with conduit headgear in accordance with one form of the present technology.

FIG. 5B shows another patient interface in the form of a nasal cradle mask with conduit headgear in accordance with another form of the present technology.

FIG. 5C shows exemplary conduit headgear in accordance with one form of the present technology.

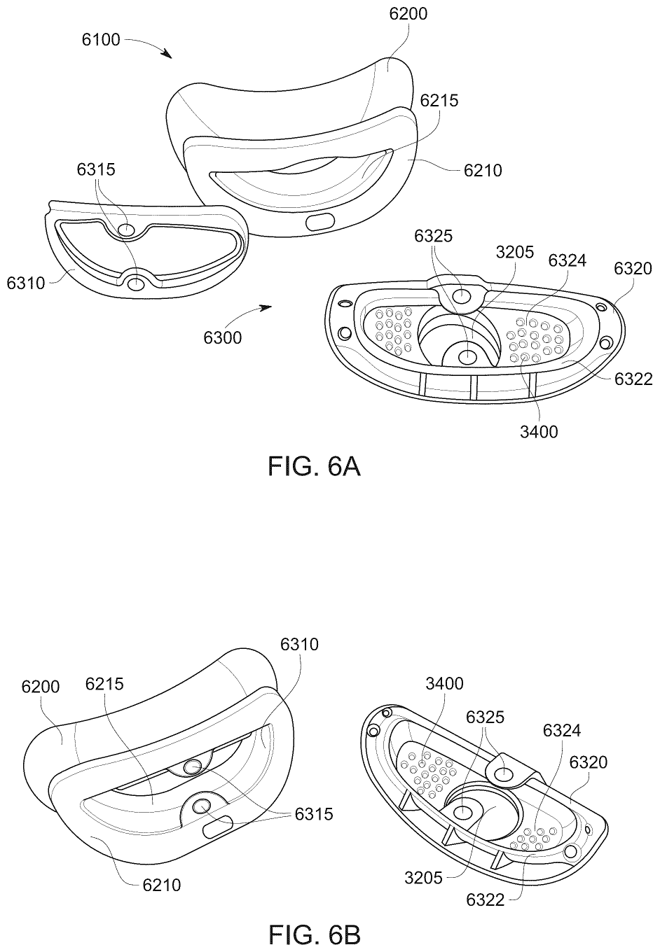

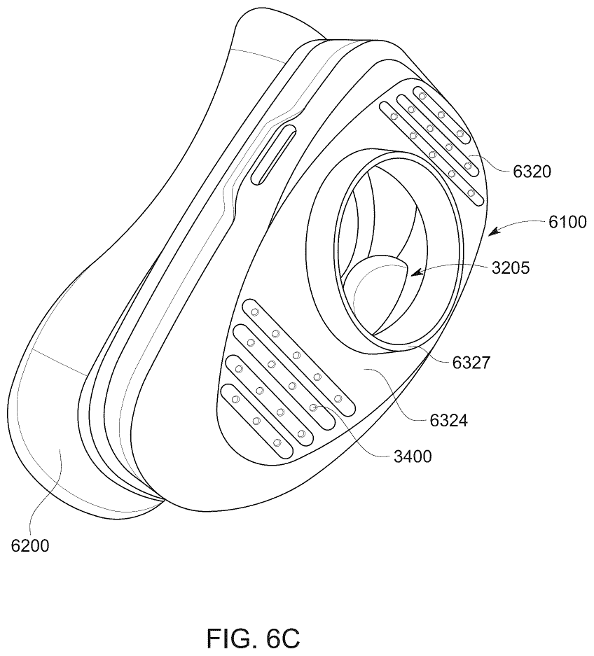

FIG. 6A shows a patient interface in a disassembled state in accordance with one form of the present technology.

FIG. 6B shows the patient interface of FIG. 6A in a partly assembled state in accordance with one form of the present technology.

FIG. 6C shows a cushion module of the patient interface of FIG. 6A in accordance with one form of the present technology.

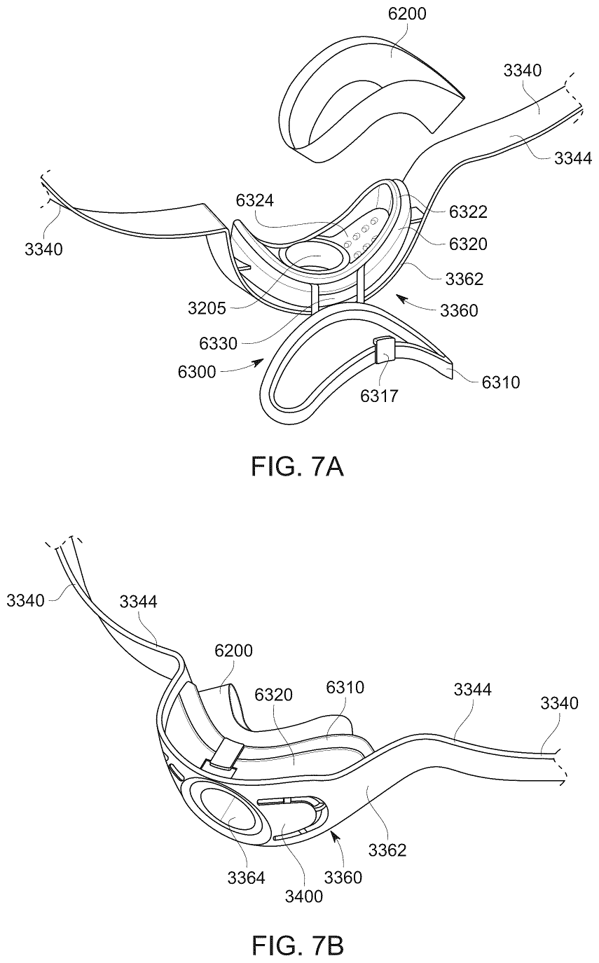

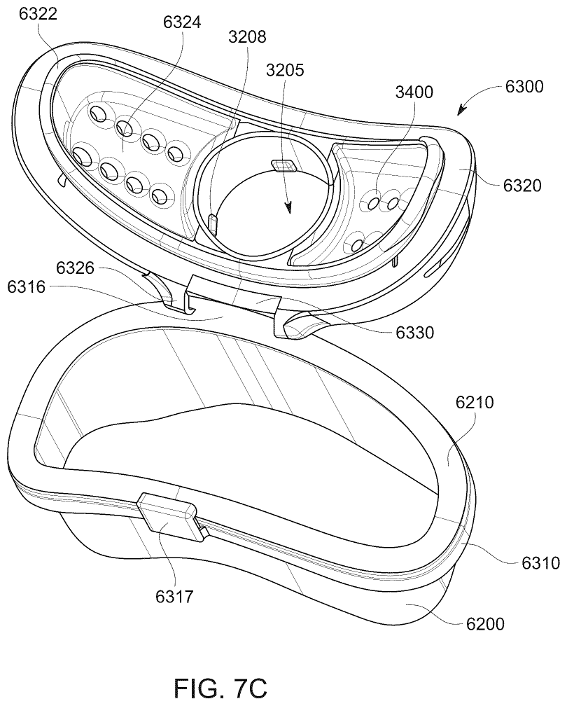

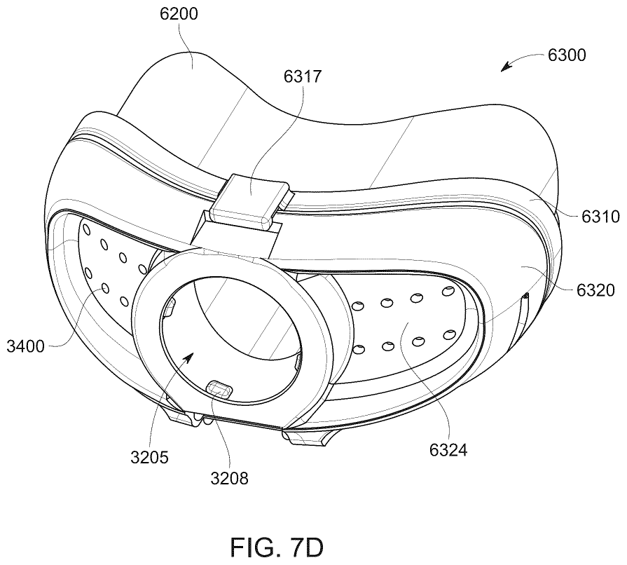

FIG. 7A shows a patient interface in a disassembled state in accordance with one form of the present technology.

FIG. 7B shows the patient interface of FIG. 7A in an assembled state in accordance with one form of the present technology.

FIG. 7C shows a cushion clip of the patient interface of FIG. 7A in an open configuration in accordance with one form of the present technology.

FIG. 7D shows a cushion clip of the patient interface of FIG. 7A in a closed configuration in accordance with one form of the present technology.

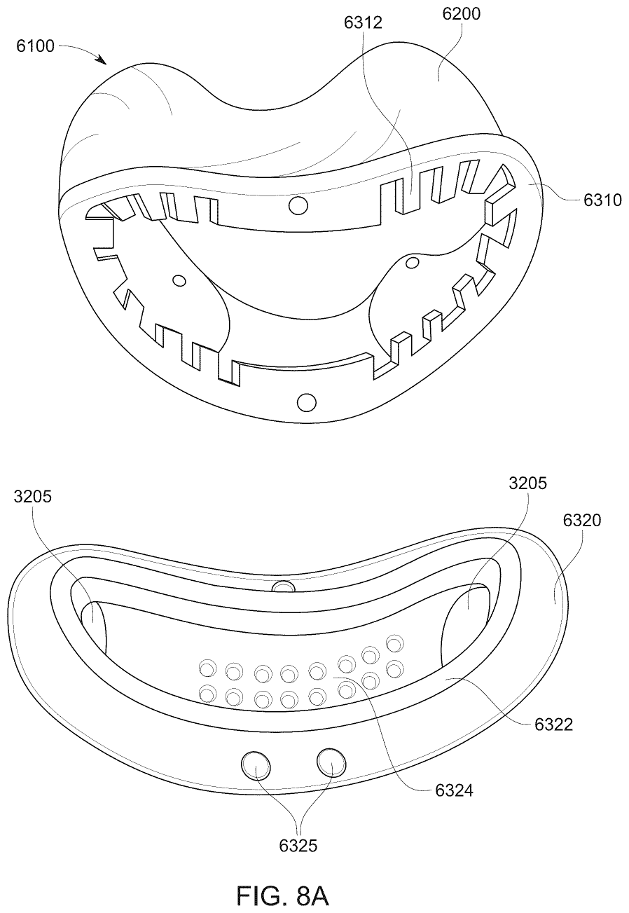

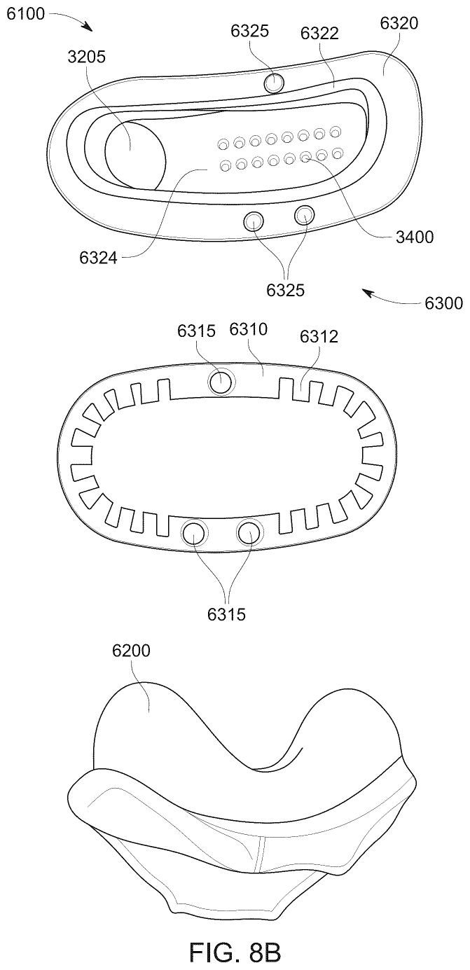

FIG. 8A shows a cushion module of a patient interface in a partly assembled state in accordance with one form of the present technology.

FIG. 8B shows the cushion module of FIG. 8B in a disassembled state in accordance with one form of the present technology.

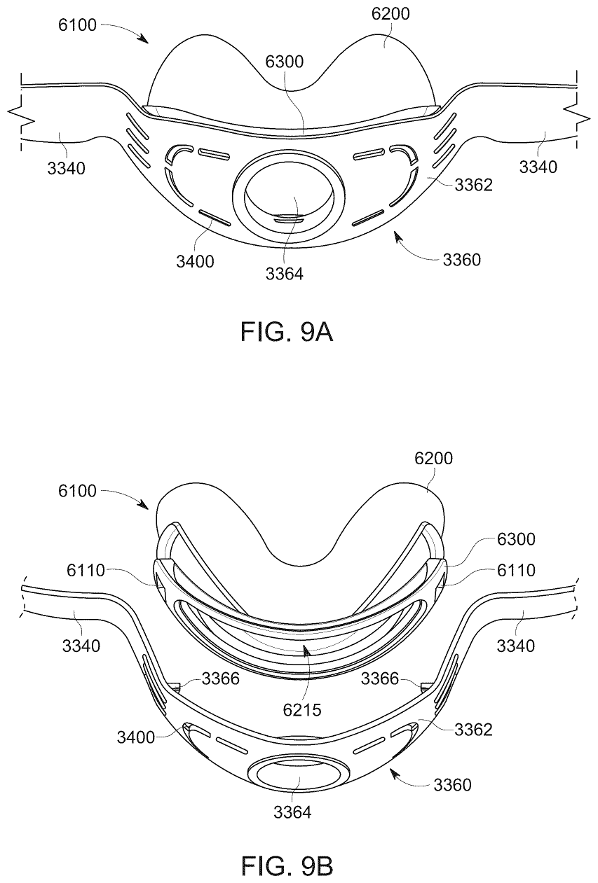

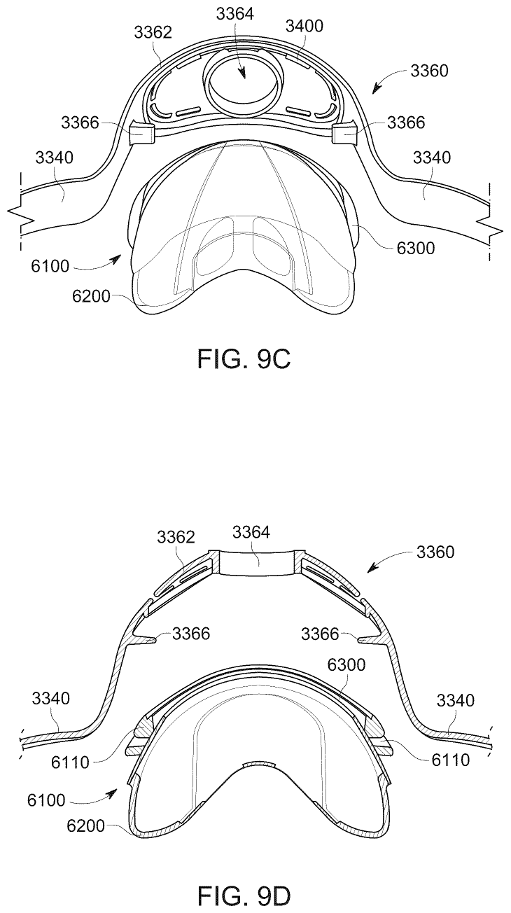

FIG. 9A shows a patient interface in accordance with one form of the present technology.

FIG. 9B shows the patient interface of FIG. 9A in a partly assembled state in accordance with one form of the present technology.

FIG. 9C shows the patient interface of FIG. 9A in a partly assembled state in accordance with one form of the present technology.

FIG. 9D shows a plan cross-sectional view of the patient interface of FIG. 9A in a partly assembled state in accordance with one form of the present technology.

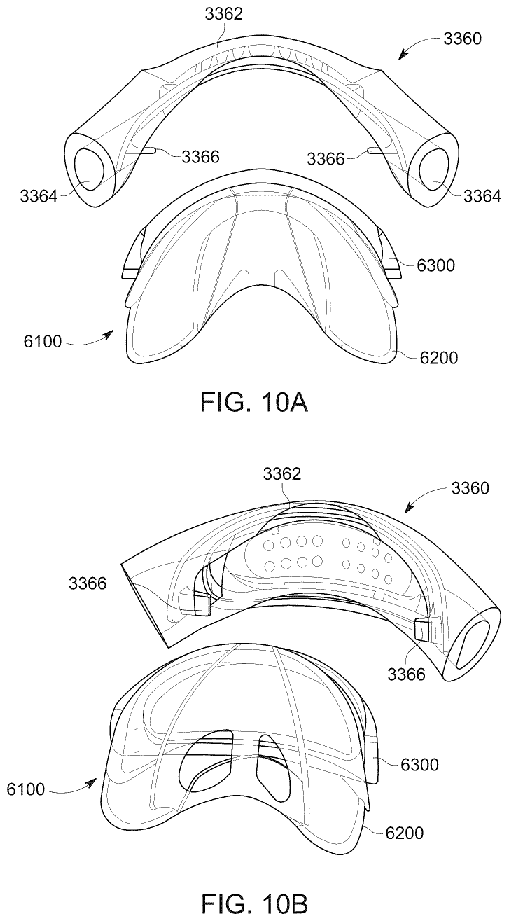

FIG. 10A shows a wireframe view of a patient interface in a partly assembled state in accordance with one form of the present technology.

FIG. 10B shows wireframe view of a the patient interface of FIG. 10A in a partly assembled state in accordance with one form of the present technology.

### 4.4 Humidifier

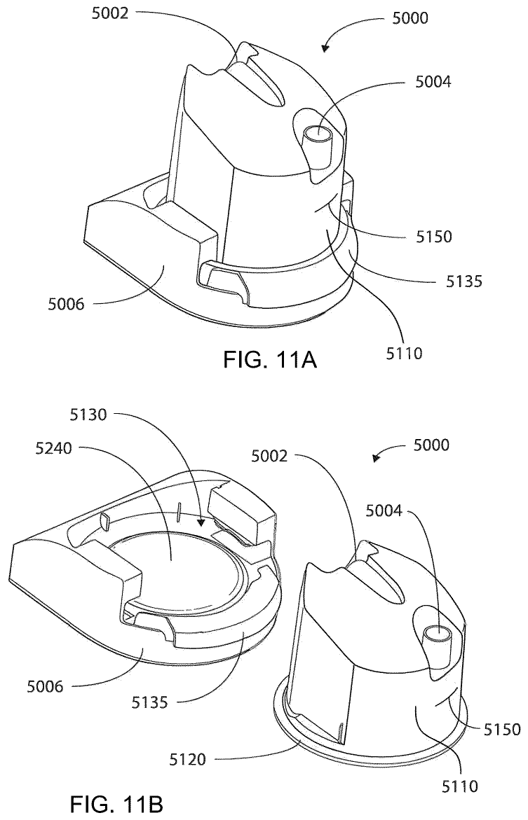

FIG. 11A shows an isometric view of a humidifier in accordance with one form of the present technology.

FIG. 11B shows an isometric view of a humidifier in accordance with one form of the present technology, showing a humidifier reservoir 5110 removed from the humidifier reservoir dock 5130.

### 5 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 5.1 Therapy

In one form, the present technology comprises a method for treating a respiratory disorder comprising applying positive pressure to the entrance of the airways of a patient 1000.

In certain examples of the present technology, a supply of breathable gas (which may be referred to as “air”) at positive pressure is provided to the nasal passages of the patient via one or both nares. In certain forms, the supply of breathable gas may additionally be provided to the mouth of the patient.

### 5.2 Respiratory Therapy Systems

In one form, the present technology comprises a respiratory therapy system for treating a respiratory disorder. The respiratory therapy system may comprise an RPT device 4000 for supplying a flow of air to the patient 1000 via an air circuit 4170 and a patient interface 3000.

### 5.3 Patient Interface

A non-invasive patient interface 3000 in accordance with certain aspects of the present technology may comprise one or more of the following functional aspects: a seal-forming structure 3100, a plenum chamber 3200, a positioning and stabilising structure 3300, a vent 3400, and a connection port 3600 for connection to air circuit 4170.

In use the seal-forming structure 3100 is arranged to form a seal with a region surrounding an entrance to the airways of the patient so as to maintain positive pressure at the entrance(s) to the airways of the patient 1000. The sealed patient interface 3000 is therefore suitable for delivery of positive pressure therapy. The seal-forming structure 3100 has an opening to allow breathable gas to be delivered to a patient's airways.

The plenum chamber 3200 is a portion of patient interface 3000 having walls at least partially enclosing a volume of space. The plenum chamber 3200 is configured to be pressurisable to therapeutic pressures, for example in the range of 4 to 30 cmH₂O with respect to ambient.

The positioning and stabilising structure 3300 is a structure configured to hold the seal-forming structure 3100 in a therapeutically effective position on the patient's head, e.g. forming a seal with a region surrounding an entrance to the patient's airways.

Connection port 3600 is an opening that allows air circuit 4170 to convey breathable gas to the plenum chamber 3200 for delivery to the patient's airways.

In some forms a functional aspect may be provided by one or more physical components. In some forms, one physical component may provide one or more functional aspects. For example, in certain forms of the technology, the functional aspects of the seal-forming structure 3100 and the plenum chamber 3200 may be provided by a physical component in the form of a cushion module 6100, which may comprise the seal-forming structure 3100 and the plenum chamber 3200. In another example, the functional aspects of the positioning and stabilising structure 3300 may be provided by physical components in the form of a frame 3360 and headgear 3302.

Further description of the functional aspects and physical component of exemplary forms of the technology will be provided in the ensuing sections.

If a patient interface is unable to comfortably deliver a minimum level of positive pressure to the airways, the patient interface may be unsuitable for respiratory pressure therapy. The patient interface 3000 in accordance with one form of the present technology is constructed and arranged to be able to provide a supply of air at a positive pressure of at least 4 cmH₂O with respect to ambient. The patient interface 3000 in accordance with one form of the present technology is constructed and arranged to be able to provide a supply of air at a positive pressure of at least 10 cmH₂O with respect to ambient. The patient interface 3000 in accordance with one form of the present technology is constructed and arranged to be able to provide a supply of air at a positive pressure of at least 20 cmH₂O with respect to ambient.

### 5.3.1 Cushion Module

Exemplary forms of patient interface 3000 are illustrated in FIGS. 3-10. The patient interface 3000 in these forms of the technology comprises a cushion module 6100. Cushion module 6100 is a physical component that comprises, or an assembly of components that together comprise, the seal-forming structure 3100 and the plenum chamber 3200.

In some forms, the cushion module 6100 is able to be readily disassembled from the other components of the patient interface 3000 as a unit by a user. In some forms, the components that together make up the cushion module 6100 may also themselves be able to be readily disassembled by a user. In this and other contexts in this specification, unless stated otherwise, the term “readily . . . <action> . . . by a user” is intended to refer to a configuration in which a user (e.g. a patient, carer or clinician) is able to perform the specified action without having to take an unusually extreme level of action. For example, in this case of “be able to be readily disassembled by a user”, this may mean that the user is able to disassemble components without having to exert an unreasonable level of force, or without having to take extreme lengths in order to do so. In particular, the term is intended to reflect a design intention for the user to be able to perform the stated action with their own faculties and by applying forces of a magnitude within a typical user's ability. In the case of something being able to be “readily disassembled by a user”, this may be contrasted with a situation in which two components are intended to be permanently assembled together, despite there existing a relatively high level of force that would, if exerted, cause the components to disassemble. Examples of configurations that may be described as, for example, readily able to be disassembled, will be provided later.

The ability to disassemble the cushion module 6100 from other parts of the patient interface 3000 may assist with the replacement of parts of the patient interface and their recyclability. Parts of the cushion module 6100 (for example, the cushion 6200) may come into contact with the patient's skin on a regular basis and/or may regularly be exposed to gases exhaled by the patient. These parts may be susceptible to getting dirty and worn out and may consequently need cleaning or replacing. If they are replaced, it would be advantageous to be able to readily recycle one or more of the parts.

In some forms, the cushion module 6100 may comprise the physical components of a cushion 6200 and a cushion clip 6300. These two physical components may together provide the functional aspects of the seal-forming structure 3100 and the plenum chamber 3200. In some forms, only the cushion 6200 may be necessary to provide either or both of these functional aspects, while the cushion clip 6300 may function to connect the cushion 6200 to other parts of the patient interface 3000. In other forms, the cushion clip 6300 may contribute to the functional aspects of the cushion module 6100.

### 5.3.1.1 Seal-Forming Structure

As explained above, in use the seal-forming structure 3100 is arranged to form a seal with a region surrounding an entrance to the airways of the patient so as to maintain positive pressure at the entrance(s) to the airways of the patient 1000.

In one form of the present technology, a seal-forming structure 3100 provides a target seal-forming region, and may additionally provide a cushioning function. The target seal-forming region is a region on the seal-forming structure 3100 where sealing may occur. The region where sealing actually occurs—the actual sealing surface—may change within a given treatment session, from day to day, and from patient to patient, depending on a range of factors including for example, where the patient interface was placed on the face, tension in the positioning and stabilising structure and the shape of a patient's face.

In one form the target seal-forming region is located on an outside surface of the seal-forming structure 3100.

In exemplary forms of the technology, the seal-forming structure 3100 forms a physical loop of material around the perimeter of the plenum chamber 3200 that, in use, contacts a closed path on the patient's face around one or more entrances to the patient's airways. The physical loop of material forms an opening 3110 through which the flow of breathable gas is delivered to the patient's airways from the plenum chamber 3200.

In certain forms of the present technology, a patient interface system is provided comprising more than one seal-forming structure 3100, each being configured to correspond to a different size and/or shape range. For example the system may comprise one form of a seal-forming structure 3100 suitable for a large-sized head, but not a small-sized head and another suitable for a small-sized head, but not a large-sized head. The plurality of seal-forming structures 3100 may differ in size and/or shape. For example, a seal-forming structure 3100 suitable for a large-sized head may form a larger opening 3110 than a seal-forming structure 3100 suitable for a small-sized head. Each of the plurality of seal-forming structure 3100 may be provided to a different cushion module 6100.

### 5.3.1.1.1 Sealing Mechanisms

In certain forms of the technology, the seal-forming structure 3100 is constructed and arranged to generally be in compression in use, e.g. as a result of elastic tension in the positioning and stabilising structure 3300 compressing the seal-forming structure 3100 against the patient's face. In some forms, some portions of the seal-forming structure 3100 may nevertheless be held in tension during use, e.g. by adjacent regions.

In certain exemplary forms of the technology, the seal-forming structure 3100 includes a sealing flange utilizing a pressure-assisted sealing mechanism. In use, the sealing flange may readily respond to a system positive pressure in the interior of the plenum chamber 3200 acting on its underside to urge it into tight sealing engagement with the face. The sealing flange may be provided to a portion of the edge of the seal-forming structure 3100 and may be configured to extend radially inwardly with respect to the physical loop of the seal-forming structure 3100 so that an inner surface of the sealing flange, i.e. a surface facing away from the patient's face, is in contact with pressurised gas in the plenum chamber 3200. The pressurised gas therefore acts on the inner surface of the sealing flange and urges the outer surface of the sealing flange into sealing engagement with the patient's face. In one form, the sealing flange extends around the entire perimeter of the edge of the seal-forming structure 3100 forming the opening. In another form, the sealing flange extends around a part of the perimeter of the edge of the seal-forming structure 3100 forming the opening. The pressure-assisted mechanism may act in conjunction with elastic tension in the positioning and stabilising structure 3300.

In another exemplary form of the technology (not illustrated), the seal-forming structure 3100 comprises a sealing flange and a support flange. The sealing flange comprises a relatively thin member with a thickness of less than about 1 mm, for example about 0.25 mm to about 0.45 mm, which extends around the perimeter of the plenum chamber 3200. Support flange may be relatively thicker than the sealing flange. The support flange is disposed between the sealing flange and the marginal edge of the plenum chamber 3200, and extends at least part of the way around the perimeter. The support flange is or includes a spring-like element and functions to support the sealing flange from buckling in use.

### 5.3.1.1.2 Regions of Sealing

In different forms of the technology, the patient interface 3000 may be configured to form a seal with different regions of a patient's face, as will now be described.

### 5.3.1.1.2.1 Nasal Pillows

In one form the seal-forming structure of the non-invasive patient interface 3000 comprises a pair of nasal puffs, or nasal pillows, each nasal puff or nasal pillow being constructed and arranged to form a seal with a respective naris of the nose of a patient.

Nasal pillows in accordance with an aspect of the present technology include: a frusto-cone, at least a portion of which forms a seal on an underside of the patient's nose, a stalk, a flexible region on the underside of the frusto-cone and connecting the frusto-cone to the stalk. In addition, the structure to which the nasal pillow of the present technology is connected includes a flexible region adjacent the base of the stalk. The flexible regions can act in concert to facilitate a universal joint structure that is accommodating of relative movement both displacement and angular of the frusto-cone and the structure to which the nasal pillow is connected. For example, the frusto-cone may be axially displaced towards the structure to which the stalk is connected.

### 5.3.1.1.2.2 Nose-Only Masks

In certain forms of the technology, for example as illustrated in FIGS. 3-10, the patient interface 3000 comprises a seal-forming structure 3100 configured to seal around an entrance to the patient's nasal airways but not around the patient's mouth. The seal-forming structure 3100 may be configured to seal to the patient's lip superior. The patient interface 3000 may leave the patient's mouth uncovered. This patient interface 3000 may deliver a supply of air or breathable gas to both nares of patient 1000 and not to the mouth. This type of patient interface may be identified as a nose-only mask.

One form of nose-only mask according to the present technology is what has traditionally been identified as a “nasal mask”, having a seal-forming structure 3100 configured to seal on the patient's face around the nose and over the bridge of the nose. A nasal mask may be generally triangular in shape. In one form, the non-invasive patient interface 3000 comprises a seal-forming structure 3100 that forms a seal in use to an upper lip region (e.g. the lip superior), to the patient's nose bridge or at least a portion of the nose ridge above the pronasale, and to the patient's face on each lateral side of the patient's nose, for example proximate the patient's nasolabial sulci. The patient interface 3000 shown in FIG. 3 has this type of seal-forming structure 3100. This patient interface 3000 may deliver a supply of air or breathable gas to both nares of patient 1000 through a single orifice.

Another form of nose-only mask may seal around an inferior periphery of the patient's nose without engaging the user's nasal ridge. This type of patient interface 3000 may be identified as a “nasal cradle” mask and the seal-forming structure 3100 may be identified as a “nasal cradle cushion”, for example. In certain forms, for example as shown in FIGS. 4-10, the seal-forming structure 3100 is configured to form a seal in use with inferior surfaces of the nose around the nares. The seal-forming structure 3100 may be configured to seal around the patient's nares at an inferior periphery of the patient's nose including to an inferior and/or anterior surface of a pronasale region of the patient's nose and to the patient's nasal alae. The seal-forming structure 3100 may seal to the patient's lip superior. The shape of the seal-forming structure 3100 may be configured to match or closely follow the underside of the patient's nose and may not contact a nasal bridge region of the patient's nose or any portion of the patient's nose superior to the pronasale. In one form of nasal cradle cushion, the seal-forming structure 3100 comprises a bridge portion dividing the opening into two orifices, each of which, in use, supplies air or breathable gas to a respective one of the patient's nares. The bridge portion may be configured to contact or seal against the patient's columella in use. Alternatively, the seal-forming structure 3100 may comprise a single opening to provide a flow or air or breathable gas to both of the patient's nares.

In some forms, a nose-only mask may comprise nasal pillows, described above.

### 5.3.1.1.2.3 Nose and Mouth Masks

In one form, the patient interface 3000 comprises a seal-forming structure 3100 configured to seal around an entrance to the patient's nasal airways and also around the patient's mouth. The seal-forming structure 3100 may be configured to seal to the patient's face proximate a chin region. This patient interface 3000 may deliver a supply of air or breathable gas to both nares and to the mouth of patient 1000. This type of patient interface may be identified as a nose and mouth mask.

One form of nose-and-mouth mask according to the present technology is what has traditionally been identified as a “full-face mask”, having a seal-forming structure 3100 configured to seal on the patient's face around the nose, below the mouth and over the bridge of the nose. A nose-and-mouth mask may be generally triangular in shape. In one form the patient interface 3000 comprises a seal-forming structure 3100 that forms a seal in use to a patient's chin-region (which may include the patient's lip inferior and/or a region directly inferior to the lip inferior), to the patient's nose bridge or at least a portion of the nose ridge superior to the pronasale, and to cheek regions of the patient's face. This patient interface 3000 may deliver a supply of air or breathable gas to both nares and mouth of patient 1000 through a single orifice. This type of seal-forming structure 3100 may be referred to as a “nose-and-mouth cushion”.

In another form the patient interface 3000 comprises a seal-forming structure 3100 that forms a seal in use on a patient's chin region (which may include the patient's lip inferior and/or a region directly inferior to the lip inferior), to an inferior and/or an anterior surface of a pronasale portion of the patient's nose, to the alae of the patient's nose and to the patient's face on each lateral side of the patient's nose, for example proximate the nasolabial sulci. The seal-forming structure 3100 may also form a seal against a patient's lip superior. A patient interface 3000 having this type of seal-forming structure may have a single opening configured to deliver a flow of air or breathable gas to both nares and mouth of a patient, may have an oral hole configured to provide air or breathable gas to the mouth and a nasal hole configured to provide air or breathable gas to the nares, or may have an oral hole for delivering air to the patient's mouth and two nasal holes for delivering air to respective nares. This type of patient interface 3000 may have a nasal portion and an oral portion, the nasal portion sealing to the patient's face at similar locations to a nasal cradle mask.

In a further form of nose and mouth mask, the patient interface 3000 may comprise a seal-forming structure 3100 having a nasal portion comprising nasal pillows and an oral portion configured to form a seal to the patient's face around the patient's mouth.

In some forms, the seal-forming structure 3100 may have a nasal portion that is separate and distinct from an oral portion. In other forms, a seal-forming structure 3100 may form a contiguous seal around the patient's nose and mouth.

It is to be understood that the above examples of different forms of patient interface 3000 do not constitute an exhaustive list of possible configurations. In some forms a patient interface 3000 may comprise a combination of different features of the above described examples of nose-only and nose and mouth masks.

### 5.3.1.1.3 Materials

In certain forms of the present technology, the seal-forming structure 3100 is constructed from a biocompatible material. A seal-forming structure 3100 in accordance with the present technology may additionally or alternatively be constructed from a soft, flexible, resilient material. Suitable materials that may be used to form the seal-forming structure 3100 in certain forms of the technology include elastomers, e.g. silicone (e.g. liquid silicone rubber (LSR)) and foams. For example, the seal-forming structure 3100 in the exemplary forms of the technology illustrated in FIGS. 3-10 is formed from LSR.

### 5.3.1.2 Plenum Chamber

As explained above, the plenum chamber 3200 is a portion of patient interface 3000 having walls at least partially enclosing a volume of space. The plenum chamber 3200 is configured to be pressurisable to therapeutic pressures, for example in the range of 4 to 30 cmH₂O with respect to ambient.

In certain forms, the volume of space enclosed by the plenum chamber 3200 may be additionally enclosed by other components of the patient interface 3000, for example the seal-forming structure 3100. In certain forms, the plenum chamber 3200 may substantially define the volume of space, which is the pressurisable volume of space into which is delivered a flow of pressurised breathable gas for breathing by the patient. That is, even though other features may form part of the boundary defining the volume of space, the plenum chamber 3200 may be considered to distinctly form a significant portion of the volume of space. In some forms, the volume of space may also be bounded by, for example, the opening in the seal-forming structure 3100 which delivers breathable gas to the patient's airways in use, or by the patient's face when the patient interface 3000 is being worn. In certain forms, the plenum chamber 3200 may comprise an anterior wall 3210 positioned on an anterior side of the plenum chamber 3200 when the patient interface 3000 is being worn. The anterior wall 3210 may have a patient-facing side that faces posteriorly towards the patient's face, and a non-patient-facing side that faces anteriorly away from the patient's face. The anterior wall 3210 may substantially form an anterior boundary of the volume of space enclosed by the plenum chamber 3200. The anterior boundary of the volume of space may also be bounded in part at or approximate the plenum chamber inlet port (described below).

In certain forms, for example the form shown in FIGS. 5A, 9 and 10, the volume of space defined by the plenum chamber 3200 may additionally be defined by the frame 3360. In such forms, the plenum chamber 3200 may not include an anterior wall, or the anterior wall may have a relatively large opening therein. Instead, when the patient interface 3000 is assembled, the plenum chamber 3200 may abut against part of the frame 3360, for example a patient-facing surface of the frame 3360, so that the frame 3360 forms an anterior boundary of the volume of space. It will be appreciated that, to reduce leaks, the plenum chamber 3200 may abut against the frame 3360 in a sealed manner.

The plenum chamber 3200 may have a perimeter that is shaped to be complementary to the surface contour of the face of an average person in the region where a seal will form in use. Alternatively, the plenum chamber 3200 may have a perimeter that is shaped to be complementary to the surface contour of the face of an actual person in the region where a seal will form in use, i.e. the plenum chamber 3200 may be customised to an individual patient. In use, a marginal edge of the plenum chamber 3200 is positioned in close proximity to an adjacent surface of the face. Actual contact with the face is provided by the seal-forming structure 3100, which is provided to the plenum chamber 3200. The seal-forming structure 3100 may extend in use about the entire perimeter of the plenum chamber 3200.

In certain forms of the technology, the plenum chamber 3200 is configured to be substantially flexible. That is, the structure of the plenum chamber 3200 and the material from which the plenum chamber 3200 is formed are such that the plenum chamber 3200 is able to be deformed. In certain forms, all of the plenum chamber 3200 is substantially flexible while in other forms a significant proportion of the plenum chamber 3200 is substantially flexible.

In certain exemplary forms of the technology, the flexibility of the plenum chamber 3200 is achieved by forming the plenum chamber 3200 from a reasonably soft material and with reasonably thin (for example a thickness of 5 mm or less) walls.

In certain forms of the present technology, the plenum chamber 3200 is constructed from a biocompatible material.

Suitable materials that may be used to form the plenum chamber 3200 in certain forms of the technology include elastomers, e.g. silicone (e.g. liquid silicone rubber (LSR)) and fabric.

In the exemplary form of the technology illustrated in FIGS. 3-10, the plenum chamber 3200 is formed from LSR. For example, the plenum chamber 3200 may be formed from an LSR having a hardness of approximately 20-50 Shore A, for example approximately 40 Shore A. A higher hardness value may increase the rigidity of the plenum chamber 3200.

In some forms of the technology, the cushion module 6100 comprising the plenum chamber 3200 and the seal-forming structure 3100 may be formed from a single homogeneous piece of material. For example, in the forms illustrated in FIGS. 3-5, the cushion module 6100 is formed from a single piece of LSR. In other forms, the plenum chamber 3200 and the seal-forming structure 3100 may both be formed from an elastomer, e.g. LSR, but have different hardnesses. For example, the plenum chamber 3200 and the seal-forming structure 3100 may be co-moulded to form the cushion module 6100.

### 5.3.1.2.1 Plenum Chamber Inlet Port

In certain forms of the technology, the plenum chamber 3200 has formed therein a plenum chamber inlet port 3205 configured to receive a flow of breathable gas for breathing by the patient. The flow of gas may be received from an inlet conduit, for example an elbow 4700. The inlet conduit may form part of air circuit 4170 or may be configured to connect to air circuit 4170.

In the forms of the technology illustrated in FIGS. 3, 4 and 6-9, the plenum chamber inlet port 3205 is located on an anterior side of the plenum chamber 3200 when the patient interface 3000 is being worn. For example, the plenum chamber inlet port 3205 may be located in the anterior wall 3210 of the plenum chamber 3200. The plenum chamber inlet port 3205 may be located so that it is positioned generally in front of an entrance to the patient's airways when the patient interface 3000 is worn.

In certain forms the plenum chamber inlet port 3205 is configured so that the inlet conduit fluidly connects to the plenum chamber inlet port 3205 in a fluidly sealed manner, for example with an interference fit. For example, the diameter of the plenum chamber inlet port 3205 may be equal to, or slightly smaller than the diameter of the inlet conduit to ensure an airtight fit. In certain forms, a gasket, for example O-ring, may be provided to the plenum chamber inlet port 3205 to help achieve a sealed connection to the inlet conduit. In the form shown in FIGS. 7A-D, the cushion clip 6300 may comprise one or more protrusions 3208 that extend radially inwardly into plenum chamber inlet port 3205, for example the protrusions may extend from the inner cylindrical surface of the cushion clip 6300 that forms the plenum chamber inlet port 3205. These protrusions may assist in helping the inlet conduit to fit into the plenum chamber inlet port 3205 with an interference fit.

### 5.3.1.3 Cushion

In certain forms, the cushion module 6100 comprises a cushion 6200. A posterior part of the cushion 6200 may form the seal-forming structure 3100, which has been described earlier. In addition, the cushion 6200 may form the plenum chamber 3200, which has also been described earlier.

In some forms, for example as shown in FIGS. 3-10, the cushion 6200 may take the form of a flexible component. For example, the cushion 6200 may be formed from an elastomer, e.g. silicone (e.g. liquid silicone rubber (LSR)) or a foam. The cushion 6200 may be a single homogeneous piece of material, for example an injection moulded piece of LSR.

In some forms, the cushion 6200 may need to be disassembled from other parts of the cushion module 6100 for cleaning and/or replacing. The cushion 6200 may be formed from a material that is relatively difficult to recycle (e.g. LSR), at least compared to parts of the patient interface 3000 that are formed from a more readily recyclable material (for example a recyclable plastic such as polycarbonate, polyethylene, polypropylene, PET or PVC, for example). Consequently, it may be advantageous to be able to completely disassemble the cushion 6200 from other parts of the patient interface 3000, e.g. the cushion module 6100.

### 5.3.1.4 Cushion Clip

In certain forms, the cushion module 6100 comprises a cushion clip 6300. The cushion clip 6300 may be configured to hold the cushion module 6100 in a substantially fixed position relative to other parts of the patient interface 3000, for example the frame 3360.

As has already been explained, in certain forms of the technology, the patient interface 3000 is configured so that the cushion module 6100 may be readily disassembled from the rest of the patient interface 3000 by a user. This feature may be enabled by the design of the cushion clip 6300, and examples of how this may be achieved will now be described.

### 5.3.1.4.1 Clip Portions

In the forms of the technology shown in FIGS. 6-8, the cushion clip 6300 comprises two clip portions: a first cushion clip portion 6310 and a second cushion clip portion 6320. The first and second cushion clip portions 6310 and 6320 may be configured to connect together when the cushion clip 6300 is in an assembled configuration, for example as shown in FIGS. 6C and 7B. The cushion clip 6300 is in an assembled configuration when the patient interface 3000 is also in an assembled configuration. In these forms, the two cushion clip portions 6310 and 6320 sandwich a portion of the cushion 6200 between them in the assembled configuration. In this configuration, the cushion clip portions are held sufficiently close together to trap and retain the cushion 6200 in place relative to the assembled cushion clip 6200. In a disassembled configuration, the two cushion clip portions 6310 and 6320 may be at least partially separated from each other, releasing the cushion 6200 from between them and allowing the cushion 6200 to be disassembled from the cushion clip 6300.

One such form of the technology in which the cushion clip 6300 comprises two cushion clip portions 6310 and 6320 is shown in FIGS. 6A-C. In this form, when the cushion clip 6300 is disassembled, the two cushion clip portions 6310 and 6320 are completely disconnected from each other.

The first cushion clip portion 6310 may take the form of a ring-shaped member that fits inside the internal volume formed by the cushion 6200. In this position, the ring-shaped member may form a loop around the plenum chamber inlet port 3205, e.g. the opening in the anterior end of the cushion 6200. The cushion 6200 may comprise a flange 6210 on an anterior side of the cushion 6200, i.e. distal from the patient's skin when the patient interface 3000 is worn. The flange 6210 may extend in a radially inward direction to form an opening 6215 in the cushion 6200 through which the flow of pressurised gas is received in use. The flange 6210 may extend around all or part of the circumference of the opening 6215. The first cushion clip portion 6310 may be shaped complementary to the shape of the flange 6210 and the opening 6215. For example, in the form of FIGS. 6A-C, these features may be oval-shaped when viewed from in front of the patient when the patient interface 3000 is assembled and worn by the patient. They may also have a curvature in one or more directions perpendicular to the plane of the ring. For example, the first cushion clip portion 6310 may be configured so that its lateral edges are curved towards the patient when in the donned position. The flange 6210 may be shaped similarly. That is, the first cushion clip portion 6310 and the flange 6210 may be curved so that a patient-facing side of either or both components is concave in the lateral direction and a non-patient-facing side is convex in the lateral direction. In other forms, these features may be another shape, for example circular (or near-circular) or any other suitable shape.

The first cushion clip portion 6310 may be configured so that it may be positioned inside the cushion 6200 and immediately posterior to the flange 6210 with the cushion clip portion 6310 abutting the patient-facing side of the flange 6210. The flange 6210 and the first cushion clip portion 6310 may be shaped so that there is close contact between the flange 6210 and the non-patient-facing side of the cushion clip portion 6310 around the circumference without creases in the flange 6210.

The second cushion clip portion 6320 may be configured to connect together with the first cushion clip portion 6310. For example, the second cushion clip portion 6320 may comprise a ring-shaped portion 6322 that is shaped complementarily to the first cushion clip portion 6310 so that the ring-shaped portion 6322 and the first cushion clip portion 6310 may be positioned close together with mating surfaces, e.g. abutting or near-abutting contact around their periphery. In the example of FIGS. 6A-C, the ring-shaped portion 6322 is substantially similar in anterior-view shape to the first cushion clip portion 6310. The ring-shaped portion 6322 may have a similar curvature relative to the plane of the ring, for example the lateral edges of the ring-shaped portion 6322 may curve towards the patient's face (referencing directions when the patient interface is donned), i.e. the ring-shaped portion 6322 may be curved so that a patient-facing side of the ring-shaped portion 6322 is concave in the lateral direction and a non-patient-facing side is convex in the lateral direction.

In use, the second cushion clip portion 6320 may be positioned immediately anterior to the flange 6210 of the cushion 6200 so that the first and second cushion clip portions sandwich the flange 6210 between them. Exemplary ways in which the first and second cushion clip portions may be held together are described below. The first and second cushion clip portions 6310 and 6320 may be separated so that the cushion 6200 may be disassembled from the cushion clip 6300.

The second cushion clip portion 6320 may further comprise a wall portion 6324. The ring-shaped portion 6322 may project outwardly from the wall portion 6324 in a posterior direction when the patient interface 3000 is assembled and worn by the patient. The wall portion 6324 may form the anterior wall 3210 of the plenum chamber 3200 described above. As shown in the figures, the wall portion 6324 may be curved so that a patient-facing side of the wall portion 6324 is concave in the lateral direction and a non-patient-facing side of the wall portion is convex in the lateral direction. The wall portion 6324 may provide a surface substantially covering the region inside the ring-shaped portion 6322. There may be gaps in the wall portion 6324 so that the region inside the ring-shaped portion 6322 is not entirely sealed, for example the wall portion 6324 may have formed therein the plenum chamber inlet port 3205, for example the plenum chamber inlet port 3205 may be located in a central region of the wall portion 6324. This form of cushion clip 6300 may be used in patient interfaces in which the flow of breathable gas is delivered to the plenum chamber 3200 via an air circuit 4170 that connects to an anterior portion of the patient interface 3000. The wall portion 6324 may also have formed therein a plurality of holes in order to allow exhaled gas to vent from the plenum chamber 3200. These holes may amount to a vent 3400 or part thereof.

Another form of the technology is shown in FIGS. 7A-D. This form has many similarities to the form of FIGS. 6A-C and the relevant parts of the earlier description will be understood to apply. The first and second cushion clip portions 6310 and 6320 of the cushion clip 6300 of FIGS. 7A-D are configured so that, in the disassembled state, the first cushion clip portion 6310 remains partly connected to the second cushion clip portion 6320. For example, the first cushion clip portion 6310 may be connected to the second cushion clip portion 6320 via a hinge 6330. In some forms, the hinge 6330 may take the form of a living hinge (i.e. a narrow neck of material integrally connected to both the first and second cushion clip portions) while in other forms, the hinge 6330 may comprise a pin inserted through aligned bores in both the first and second cushion clip portions 6310 and 6320 in order to rotatably connect the two cushion clip portions. In such forms, the first and second cushion clip portions 6310 and 6320 may comprise respective protruding members 6316 and 6326 through which are the bores, the protruding members being able to be positioned relative to each to align the bores, as shown in the example of FIG. 7C. Other forms of hinge may be used in other forms of the technology. The first and second cushion clip portions may move relative to each other by virtue of hinge 6330 so that, in an assembled configuration, as shown in FIGS. 6B and 6D, mating surfaces of the respective cushion clip portions come close together and sandwich the cushion 6200, for example the flange 6210 of the cushion 6200, between them. Further, in a disassembled configuration, the cushion clip portions 6310 and 6320 are partially separated so that the cushion 6200 is released and able to be disassembled but the first and second cushion clip portions remain partly connected at the hinge 6330.

The hinge 6330 may be located at any point around the periphery of the cushion clip 6300. In the form shown in FIGS. 7A-D, the hinge 6330 is located at a substantially central region of the superior or inferior edge of the cushion clip 6300.

Another difference between the form of technology shown in FIGS. 7A-D compared to the form shown in FIGS. 6A-C is that, in FIGS. 6A-C, the first cushion clip portion 6310 is positioned radially inside a wall of the cushion 6200 in the assembled configuration whereas, in FIGS. 7A-D, the first cushion clip portion 6310 is positioned radially outside a wall of the cushion 6200 and encircles an outer periphery of an external surface of the cushion 6200 (as shown most clearly in FIG. 7D). In the form of FIGS. 7A-D, the cushion 6200 may comprise a flange 6210 that extends radially outwardly from the opening 6215 in the anterior end of the cushion 6200. To assemble the cushion module 6100 in this form, the cushion 6200 may be pushed through the opening in the middle of the ring-shaped first cushion clip portion 6310 until the first cushion clip portion 6310 abuts against the patient-facing side of the flange 6210.

In the form of technology shown in FIG. 7D, parts of the wall portion 6324 may be recessed from an anterior side of the cushion clip 6300. For example, the parts of the wall portion 6324 in which the holes forming vent 3400 are positioned may be recessed from the anterior side of the cushion clip 6300. When the cushion clip 6300 is assembled with the frame 3360, this recess may result in there being a gap between the vent holes in the wall portion 6324 and the vent holes in the frame 3360. In some forms, this gap may assist in reducing the level of noise when venting exhaled air and/or with dispersing the vented air and reducing the strength of jets of air that form, which may improve comfort, if the jets of air are directed towards the patient or their bed partner, and reduce noise, if the jets of air are directed towards an object such as the bed clothes.

Another form of the technology is shown in FIGS. 8A-B. This form has many similarities to the forms of FIGS. 6A-C and FIGS. 7A-D and the relevant parts of the earlier description will again be understood to apply. In this form, the first and second cushion clip portions 6310 and 6320 are again able to be completely separated in the disassembled configuration.

In the form of FIGS. 8A-B, the first cushion clip portion 6310 comprises a ring-shaped member with a plurality of teeth 6312 projecting radially inwardly. The teeth 6312 may be formed integrally with the ring-shaped member of the first cushion clip portion 6310, for example the first cushion clip portion 6310 may be formed as a single moulded member. The teeth 6312 may take the form of protrusions spaced apart from each other around the interior periphery of the ring-shaped member. When the cushion clip 6300 is assembled with the cushion 6200 to form the cushion module 6100, the teeth 6312 may assist the cushion clip 6300 to grip against the interior walls of the cushion 6200 in order to hold the cushion 6200 in place relative to the cushion clip 6300. While, in the form shown in FIGS. 8A-B, the teeth 6312 are provided substantially around the entire interior periphery of the first cushion clip portion 6310, in other forms, the teeth 6312 may be provided around only a part of the entire periphery of the first cushion clip portion 6310. In other forms, the teeth 6312 may project outwardly from the ring-shaped member in a different direction, for example anteriorly, in order to grip the cushion 6200. In the form shown in FIGS. 8A-B, the teeth 6312 may comprise substantially square-shaped tips and the corners of the tips may act to grip the interior wall of the cushion 6200. In other forms, the teeth 6312 may be pointed (e.g. triangular) or they may have a rounded head portion, and in some forms a head portion that is wider than a neck portion. In other forms, the teeth 6312 may be hook-shaped. Any of these shapes may grip the interior surface of the cushion 6200. In some forms a rounded tip to the teeth 6312 may be advantageous to reduce the risk of the teeth tearing the cushion 6200.

In the forms of technology shown in FIGS. 6 and 7, the cushion clip 6300 may comprise a single plenum chamber inlet port 3205 and this may be located in a central region of the wall portion 6324 of the second cushion clip portion 6320. In other forms, for example the form of the technology shown in FIGS. 8A-B, the cushion clip 6300 may comprise two plenum chamber inlet ports 3205. These plenum chamber inlet ports 3205 may be formed in the wall portion 6324 of the second cushion clip portion 6320. A first of the plenum chamber inlet ports 3205 may be located on a first lateral side of the plenum chamber 6200, e.g. on a first lateral side of the wall portion 6324, and a second of the plenum chamber inlet ports 3205 may be located on a second lateral side of the plenum chamber 6200, e.g. on a second lateral side of the wall portion 6324. This form of cushion clip 6300 may be used in patient interfaces in which the flow of breathable gas is delivered to the plenum chamber 3200 via headgear tubes 3350, as will be described later.

It should be appreciated that, while the form of cushion clip 6300 shown in FIGS. 8A-B has two plenum chamber inlet ports 3205 and the forms of the cushion clip 6300 shown in FIGS. 6-7 have a single plenum chamber inlet port 3205, in other forms, a cushion clip 6300 similar to that shown in FIGS. 6-7 may comprise two plenum chamber inlet ports 3205 and a cushion clip 6300 similar to that shown in FIGS. 8A-B may comprise a single plenum chamber inlet port 3205. That is, these features may be able to be mixed and matched between the exemplary forms of the technology.

In certain forms, the first and/or second cushion clip portions 6310 and 6320 may be formed from a material that may be readily recycled, e.g. may be recycled through common recycling processes or channels accessible to many households. For example, the cushion clip portions may be formed from a recyclable plastic such as polycarbonate, polyethylene, polypropylene, PET or PVC, for example.

### 5.3.1.4.2 Inter-Connection Between Cushion Clip Portions

It has been explained that the first and second cushion clip portions 6310 and 6320 may be configured such that they are able to be held together with a part of the cushion 6200 sandwiched between them. In certain forms, for example those shown in FIGS. 6 and 8, the first cushion clip portion 6310 may be magnetically connected to the second cushion clip portion 6320 when the clip portions are assembled together. For example, the first cushion clip portion 6310 may comprise one or more first magnetic members 6315. In the forms shown, the first magnetic member(s) 6315 may be positioned on, or embedded in, a surface of the first cushion clip portion 6310 facing away from the patient during use, and facing towards the second cushion clip portion 6320. In another form, the first magnetic member(s) 6315 may be embedded inside the first cushion clip portion 6310. Similarly, the second cushion clip portion 6320 may comprise one or more second magnetic members 6325. In the forms shown, the second magnetic member(s) 6325 may be positioned on, or embedded in, a surface of the second cushion clip portion 6320 facing towards the patient during use, and facing towards the first cushion clip portion 6310. In another form, the second magnetic member(s) 6325 may be embedded inside the second cushion clip portion 6320. The first magnetic member(s) 6315 may be positioned on or in the first cushion clip portion 6310 in positions so that, when the second cushion clip portion 6320 is brought into mating engagement with the first cushion clip portion 6310, the respective first and second magnetic members come into magnetic engagement with each other.

Using a plurality of magnetic members on each of the first and second cushion clip portions may be advantageous in promoting a better retention of the first and second cushion clip portions together than if only a single magnetic member is provided to each cushion clip portion. The greater the number of magnetic members used, the better the connection between the two cushion clip portions and the reduced risk of pressurised gas leaking out of the cushion module 6100. On the other hand, the more magnetic members that are used, the greater the complexity and cost of the design and assembly of the cushion module 6100. In some forms, the plurality of magnetic members may be distributed around the periphery of the ring-shaped first cushion clip portion 6310 and the ring-shaped member 6322 of the second cushion clip portion 6320. In the forms shown, a first pair of connecting magnetic members may be provided in a medial region of a superior region of the first and second cushion clip portions, and a second pair of connecting magnetic members may be provided in a medial region of an inferior region of the first and second cushion clip portions.

It will be understood that magnetic members are members that are able to connect to another magnetic member through magnetic attraction. For each pair of magnetic members configured to connect together, one or both of the magnetic members may be a magnet, for example a permanent magnet. In forms in which only one of the pair of magnetic members is a permanent magnet, the other magnetic member of the pair is formed from a magnetic material, i.e. a material that is attracted to a permanent magnet, for example a ferromagnetic material such as iron, nickel, etc.

The magnetic members may be configured so that the magnitude of magnetic attraction between the magnetic members when they are connected together is sufficient to maintain connection between the members during typical use of the patient interface 3000 to administer respiratory therapy. For example, the magnetic members are configured so that they are not disconnected when the plenum chamber 3200 is pressurised to typical therapeutic pressures, e.g. up to 20 to 30 cmH₂O. In addition, the magnitude of the magnetic attraction is not so strong that a user cannot disconnect the magnetic members given application of a disconnection force that is achievable manually by a typical user, or with the aid of an everyday tool. That is, the first and second cushion clip portions 6310 and 6320 may be able to be readily disconnected by a user by overcoming the magnetic attraction between the magnetic members.

In certain forms, the first and second cushion clip portions 6310 and 6320 may be configured such that the first and second magnetic members 6315 and 6325 may be readily removed from the respective cushion clip portions by a user. For example, in some forms, the first and second cushion clip portions 6310 and 6320 may have recesses formed in their surface where the respective magnetic members are to be located and the magnetic members may be mounted on the cushion clip portions by press fitting the magnetic members into the recesses. The magnetic members may then be removed by pulling the magnetic members out of the recesses (or by pushing them out by application of force to the opposing side of the cushion clip portions). Being able to readily separate the magnetic members from the cushion clip portions in this way may help to dispose of the cushion clip 6300 in a way which promotes recycling. For example, the cushion clip portions may be formed from a material that is readily recyclable, for example from a recyclable plastic, while the magnetic members may be more difficult to recycle, or may be recycled through a separate process and consequently need to be disassembled form the cushion clip portions.

In other forms, an alternative mechanism may be used to connect the first and second cushion clip portions 6310 and 6320 in the assembled configuration. For example, in some forms, the first and second cushion clip portions may be held together by an interlock mechanism. One example is shown in FIG. 7, in which the first cushion clip portion 6310 comprises a hook 6317 which is configured to interlock with a part of the second cushion clip portion 6320 when the two cushion clip portions are brought together. For example, the hook 6317 may be configured to interlock with an edge of the wall portion 6324 of the second cushion clip portion 6320. In other forms, the hook 6317 may be provided on the second cushion clip portion 6320 and the hook may be configured to interlock with a part of the first cushion clip portion 6310 when the cushion clip portions are brought together. In some forms, a plurality of interlocks (e.g. hooks) may be used.

In other forms of the technology, the connection between the first and second cushion clip portions 6310 and 6320 may be achieved through a friction-fit mechanism. For example, the ring-shaped portion 6322 of the second cushion clip portion 6320 may be configured to fit sufficiently tightly within or outside the first cushion clip portion 6310 to hold the two cushion clip portions together through friction. Sufficient tolerance in the relative size of the interfering parts of the cushion clip portions may be provided to allow a part of the cushion 6200, e.g. flange 6210, between the two cushion clip portions.

### 5.3.1.4.3 Single Cushion Clip

In other forms, for example as shown in FIGS. 9 and 10, the cushion module 6100 may comprise a cushion clip 6300 that comprises only a single part. That part may be a ring-shaped member of a similar form to that described above of the first cushion clip portion 6310 in some forms of the technology.

In some such forms, the cushion 6200 may connect to the cushion clip 6300 by a press-fit mechanism. For example, in the form shown in FIGS. 9 and 10, the anterior end of the cushion 6200, i.e. the ring-shaped portion of the cushion 6200 surrounding the plenum chamber inlet port 3205 may be configured to adopt an interference fit within a channel in a patient-facing side of the cushion clip 6300. In other such forms, the cushion 6200 and cushion clip 6300 may be integrally formed, for example the cushion clip 6300 may be overmoulded onto the cushion 6200, or vice versa. However, it may be advantageous to use a connection mechanism that allows the cushion 6200 to be readily disassembled from (and, if necessary, re-assembled to) the cushion clip 6300 by a user for ease of cleaning, replacement and disposal. In particular, if the cushion 6200 and cushion clip 6300 are formed from different materials which are recycled using different processes, recycling the components is easier if they are readily separable by a user.

### 5.3.1.5 Vent

In certain forms of the technology, the patient interface 3000 includes a vent 3400 constructed and arranged to allow for the washout of exhaled gases, e.g. carbon dioxide, from the plenum chamber 3200.

In certain forms the vent 3400 is configured to allow a continuous vent flow from an interior of the plenum chamber 3200 to ambient whilst the pressure within the plenum chamber is positive with respect to ambient. The vent 3400 may be configured such that the vent flow rate has a magnitude sufficient to reduce rebreathing of exhaled CO₂ by the patient while maintaining the therapeutic pressure in the plenum chamber in use.

In certain forms, the vent 3400 may be provided to the cushion module 6100, for example comprised as part of plenum chamber 3200. For example, the vent 3400 may be located in an anterior wall portion 6324 of the plenum chamber. In the example shown in FIGS. 6-10, the vent 3400 comprises a plurality of holes formed in the second cushion clip portion 6320 of the cushion clip 6300.

In certain forms, the vent 3400 takes the form of a plurality of through-holes in a surface of the plenum chamber, for example, about 20 to about 80 holes, or about 40 to about 60 holes, or about 45 to about 55 holes.

In other forms, the vent 3400 may be located in another part of the patient interface 3000 or respiratory therapy system, such as the inlet conduit or elbow, as described further by way of example below.

### 5.3.2 Positioning and Stabilising Structure

The seal-forming structure 3100 of the patient interface 3000 of the present technology may be held in a therapeutically effective position on the patient's head in use, e.g. in a position in which the seal-forming structure 3100 is sealed against the patient's face, by the positioning and stabilising structure 3300.

In certain forms the positioning and stabilising structure 3300 provides a retention force at least sufficient to overcome the effect of the positive pressure in the plenum chamber 3200 to lift off the face.

In certain forms the positioning and stabilising structure 3300 provides a retention force to overcome the effect of the gravitational force on the patient interface 3000.

In certain forms the positioning and stabilising structure 3300 provides a retention force in excess of the minimum retention force that may be required to maintain the seal-forming structure 3100 in position as a safety margin to overcome the potential effect of disrupting forces on the patient interface 3000, such as from tube drag, or accidental interference with the patient interface.

In certain forms, the positioning and stabilising structure 3300 comprises a frame 3360 and headgear 3302.

### 5.3.2.1 Frame

In certain forms of the technology, the patient interface 3000 comprises a frame 3360. The frame 3360 may be considered to be part of the positioning and stabilising structure 3300 since it functions to help hold the seal-forming structure 3100 in a therapeutically effective position on the patient's head. The frame 3360 may also provide structural rigidity to the patient interface 3000.

### 5.3.2.1.1 Frame Body

In certain forms of the technology, the frame 3360 comprises a frame body 3362. The frame body 3362 may be the main body of material forming the frame 3360. In certain forms, the frame body 3362 may be constructed to possess rigidity, e.g. a resistance to bending in at least one direction. It will be appreciated that both the shape of the frame body 3362 and the material from which it is made may contribute to the rigidity. In some forms, the frame body 3362 is formed from a relatively hard material and/or a material that is relatively inelastic. In some forms, the frame body 3362 may be made from a rigid plastic, e.g. polycarbonate.

In the forms of the technology illustrated in FIGS. 7, 9 and 10, the frame body 3362 takes the form of an elongate body that is wider in the lateral direction than it is tall in the superior-inferior direction (based on the intended orientation of the frame body 3362 when the patient interface 3000 is worn). The frame body 3362 may have a lateral extent substantially similar to the lateral extent of the cushion 6200, and the frame body 3362 may also be configured to substantially cover the cushion module 6100 from the perspective of a viewer standing in front of the patient when the patient is wearing the patient interface 3000. In some forms, for example as shown in FIGS. 7, 9 and 10, the frame body 3362 may be curved so that a patient-facing side of the frame body 3362 is concave in the lateral direction and a non-patient-facing side of the frame body 3362 is convex in the lateral direction.

The frame 3360 may facilitate fluid connection of the air circuit 4170 to the plenum chamber 3200. In some forms, the frame body 3362 may comprise an opening 3364 through which a flow of breathable gas may be conveyed from the air circuit 4170 to the plenum chamber inlet port 3205. As shown in the exemplary form of FIG. 9C, the frame body 3362 may comprise a collar surrounding the opening 3364. In the form shown, the collar projects outwardly on a patient-facing side of the frame body 3362 towards the patient in use. In other forms, the collar may project outwardly on a non-patient-facing side of the frame body 3362 away from the patient in use. In still other forms, the frame body 3362 may comprise collars extending in both directions.

In the form of the technology shown in FIGS. 7 and 9, the opening 3364 may be configured so that the air circuit 4170 may connect to a non-patient-facing side of the opening 3364 in order to deliver the supply of breathable gas. The cushion module 6100 may connect to the frame 3360, for example in any of the ways described earlier, with the plenum chamber 3200 in fluid connection with the opening 3364 so that the flow of breathable gas passes through the frame 3360 into the plenum chamber 3200 through the plenum chamber inlet port 3205. The opening 3364 may be positioned substantially centrally in the frame body 3362 so that, when the patient interface 3000 is worn, the opening 3364 is positioned substantially in the mid-sagittal plane of the patient's body.

In the form of technology shown in FIGS. 10A-B, the frame body 3362 comprises an elongate hollow body with openings 3364 at each lateral end. The frame body 3362 may be configured so that headgear tubes 3350 of conduit headgear are able to connect to the frame body 3362 in order to deliver the flow of breathable gas from the air circuit 4170 to the internal volume of the frame body 3362. In this form, the frame body 3362 may comprise an additional opening on a patient-facing side for the flow of breathable gas to be delivered to the cushion module 6100 through the plenum chamber inlet port 3205.

In certain forms, the frame body 3362 may be configured to flex in one or more directions. In order for the frame body 3362 to provide some rigidity to the patient interface 3000, the frame body 3362 may be configured to flex in one direction but be relatively rigid in the other direction. For example, in the case of the frame body 3362 illustrated in FIGS. 7, 9 and 10, the frame body 3362 may be able to bend relatively easily when a force is applied to the lateral edges of the frame body 3362 in order to push the lateral edges of the frame body 3362 towards or away from the patient's face. In contrast, the frame body 3362 in these forms may be difficult to bend when a force is applied to the superior and inferior edges of the frame body 3362 in order to push the superior and inferior edges of the frame body 3362 towards or away from the patient's face. This property may be held by the frame body 3362 due to its relative dimensions. For example, the frame body 3362 may be longer in the lateral direction than in the superior-inferior direction. In addition, the presence of rigidiser arms 3340 may act as lever arms to facilitate bending of the frame body 3362 by movement of the lateral edges. An example of an advantage of this property will be described later in relation to the forms shown in FIGS. 9A-D and 10A-B.

In certain forms, for example the form shown in FIGS. 10A-B, the frame body 3362 may comprise vent 3400. For example, the frame body 3362 may comprise a plurality of holes on a non-patient-facing side, for example the anterior surface of the frame body 3362.

### 5.3.2.1.2 Headgear Connectors

In certain forms, the frame 3360 further comprises two or more headgear connectors for connecting to headgear 3302. The headgear connectors may, in certain forms be provided to lateral sides of the frame body 3362, or may be positioned laterally from the frame body. For example, in the examples of FIGS. 7, 9 and 10, the headgear connectors may each be located at a distal end of an arm 3340 that extends laterally outward from the frame body 3362. In certain forms, the arm 3340 may be a rigidiser arm 3340, as described below.

The headgear connectors may be any component or part of the frame 3360 that is able to connect to headgear 3302 with sufficient rigidity that, when the patient interface 3000 is worn by a patient, the connection between the frame 3360 and the headgear 3302 is able to maintain the seal-forming structure 3100 in a therapeutically effective position on a patient's face. In certain forms, the headgear connectors may comprise hooks or holes in the frame body 3362 to which a headgear strap may attach, for example by being threaded through the hole and connected to itself using a hook-and-loop fastener. In other forms, the headgear connectors may comprise one part of a clip mechanism, where the mating part of the clip mechanism is provided at the end of headgear straps. In other forms, the headgear connectors may comprise a magnetic member to which another magnetic member in the headgear straps may be able to connect. In the form shown in FIGS. 10A-B, the headgear connectors may each comprise an open-ended tube at the lateral sides of the frame body 3360. Headgear 3302 in the form of headgear tubes 3350 may be able to connect to the tubes by a suitable connection, for example friction fit over the ends of the tubes. Other forms of headgear connector will be understood to the skilled addressee and may be used in other forms of the technology.

### 5.3.2.1.3 Rigidiser Arm

As shown in FIGS. 7 and 9, a rigidiser arm 3340 may be an elongated, rigid member that assists in maintaining the cushion 6200 in an operating position. In some forms, the rigidiser arm 3340 may contact a side of the patient's head and provide a force to limit slipping of the seal-forming structure 3100 from the patient's nose and/or mouth.

In some forms, the rigidiser arm 3340 is constructed from a rigid material (e.g., plastic). The rigid material may not permit the rigidiser arm 3340 to stretch. Additionally, the rigidiser arm 3340 may be substantially inflexible and may be unable to bend in at least one direction. The rigidiser arm 3340 may be pre-molded into a desired shape in order to fit a patient's head. For example, the rigidiser arms 3340 may be molded with a curved shape to substantially correspond to the shape of the side of the patient's head (e.g., overlaying the masseter muscle and/or the temporal bone).

In certain forms, the rigidiser arm 3340 may be molded in order to conform to a specific patient's head (e.g., the rigidiser arm 3340 is customized).

In some forms, the rigidiser arm 3340 may be flexible along at least one direction. For example, the rigidiser arm 3340 may be flexible about its width and may be inflexible along its length. In other words, the rigidiser arm 3340 may be bendable about an axis spanning the width of the rigidiser arm 3340, but may be unable to bend about an axis perpendicular to the rigidiser arm 3340. This may allow an individual patient to adjust the rigidiser arm 3340 in order to better fit their individual head.

In certain forms, the rigidiser arm 3340 may remain in a new position after being bent. This may allow a patient adjust the shape of the rigidiser arm 3340 for their specific head and then the rigidiser arm 3340 will keep the desired shape while in use in order to promote patient comfort.

In some forms, a distal end 3342 of the rigidiser arm 3340 may be a free end and a proximal end 3344 (e.g., opposite of the distal end 3342) of the rigidiser arm 3340 may be fixed. The distal end 3342 may be curved in order to minimize sharp edges that could cause patient discomfort. When the patient interface 3000 is worn, in some forms the distal end 3342 may overlay the patient's head proximate to the temporal bone, while in other forms the distal end 3342 may overlay the patient's cheek region. The proximal end 3344 may be fixed to the frame body 3362. In certain forms, for example as shown in FIGS. 7 and 9, the frame body 3362 and rigidiser arms 3340 may be integrally connector. For example, they may be moulded as a single unitary piece.

### 5.3.2.2 Headgear

In certain forms, the positioning and stabilising structure 3300 comprises headgear 3302. Headgear 3302 may be worn on the patient's head, and may particularly be designed to be worn over or around superior or posterior regions of the patient's head, to help maintain the seal-forming structure 3100 in the desired position at the anterior of the patient's head.

### 5.3.2.2.1 Straps

In certain forms of the technology, the positioning and stabilising structure 3300 comprises at least one strap 3310. The strap 3310 may be flat, i.e. relatively thin compared to its length and height, and may also be bendable and non-rigid, so that it can be worn comfortably when the patient is sleeping, including lying on the strap 3310.

The strap 3310 may be configured to be arranged, in use, so that the strap 3310 connects to the frame 3360 and passes around the back and/or top of the patient's head. When worn, the strap 3310 may be under tension to apply a retention force on the cushion module 6100 towards the patient's face, e.g. in a generally posterior direction, in order to hold the cushion module 6100 in an operative position.

The strap 3310 may be formed from one or more materials. In some forms in which the strap 3310 is formed from a plurality of materials, the materials may be blended, for example woven, together to form a single blended material. In other forms in which the strap 3310 is formed from a plurality of materials, the strap may comprise a plurality of portions, each formed from a different material, that are connected together to form the strap 3310.

In some forms of the headgear 3302, the strap 3310 may be constructed from a textile material, which may be comfortable against the patient's skin. The textile may be flexible in order to conform to a variety of facial contours. Although the textile may include rigidisers along a selected length, which may limit bending, flexing, and/or stretching of the headgear 3302.

In certain forms, the strap 3310 may be at least partially extensible. For example, the strap 3310 may include elastic, or a similar extensible material. For example, the entire strap 3310 may be extensible or selected portions may be extensible (or more extensible than surrounding portions). This may allow the strap 3310 to stretch while under tension, which may assist in providing a sealing force for the seal-forming structure 3100.

In some forms the headgear 3302 may be a two-point connection headgear. This means that the headgear 3302 may connect to the frame at two separate places, i.e. one on either side of the patient's head. Other forms of patient interface 3000 may comprise other forms of headgear 3302, for example four-point connection headgear 3302.

### 5.3.2.2.2 Conduit Headgear

In some forms of the present technology, the positioning and stabilising structure 3300 comprises one or more headgear tubes 3350 that deliver pressurised air received from a conduit forming part of the air circuit 4170 from the RPT device to the patient's airways, for example through the plenum chamber 3200 and seal-forming structure 3100. In the forms of the present technology illustrated in FIG. 5 and configured to assemble with the form illustrated in FIG. 10, the positioning and stabilising structure 3300 comprises two tubes 3350 that deliver air to the plenum chamber 3200 from the air circuit 4170. The tubes 3350 are configured to position and stabilise the seal-forming structure 3100 of the patient interface 3000 at the appropriate part of the patient's face (for example, the nose and/or mouth) in use. This allows the conduit of air circuit 4170 providing the flow of pressurised air to connect to a connection port 3600 of the patient interface in a position other than in front of the patient's face, for example on top of the patient's head. In the form of FIG. 10, the tubes 3350 (not illustrated) are configured to deliver air to openings 3364 on either lateral side of the frame body 3362.

In the forms of the present technology illustrated in FIGS. 5A-C, the positioning and stabilising structure 3300 comprises two tubes 3350, each tube 3350 being positioned in use on a different side of the patient's head and extending across the respective cheek region, above the respective ear (superior to the otobasion superior on the patient's head) to the elbow 3610 on top of the head of the patient 1000. This form of technology may be advantageous because, if a patient sleeps with their head on its side and one of the tubes 3350 is compressed to block or partially block the flow of gas along the tube 3350, the other tube 3350 remains open to supply pressurised gas to the patient. In other examples of the technology, the patient interface 3000 may comprise a different number of tubes, for example one tube, or two or more tubes.

In one example in which the patient interface has one tube 3350, the single tube 3350 is positioned on one side of the patient's head in use (e.g. across one cheek region) and a strap forms part of the positioning and stabilising structure 3300 and is positioned on the other side of the patient's head in use (e.g. across the other region) to assist in securing the patient interface 3000 on the patient's head. For example, the tube 3350 and the strap may each be under tension in use in order to assist in maintaining the seal-forming structure 3100 in a sealing position.

In the forms of the technology shown in FIGS. 5A-C, the two tubes 3350 are fluidly connected at superior ends to each other and to the connection port 3600. In some examples, the two tubes 3350 are integrally formed while in other examples the tubes 3350 are formed separately but are connected in use and may be disconnected, for example for cleaning or storage. Where separate tubes are used, they may be indirectly connected together, for example each may be connected to a T-shaped connector. The T-shaped connector may have two arms/branches each fluidly connectable to a respective one of the tubes 3350. Additionally, the T-shaped connector may have a third arm or opening providing the connection port 3600 for fluid connection to the air circuit 4170 in use. The opening may be an inlet 3332 (see e.g., 5C) for receiving the flow of pressurized air.

The tubes 3350 may be formed from a flexible material, such as an elastomer, e.g. silicone or TPE, and/or from one or more textile and/or foam materials. The tubes 3350 may have a preformed shape and may be able to be bent or moved into another shape upon application of a force but may return to the original preformed shape in the absence of said force. The tubes 3350 may be generally arcuate or curved in a shape approximating the contours of a patient's head between the top of the head and the nasal or oral region.

Each tube 3350 may be configured to receive a flow of air from the connection port 3600 on top of the patient's head and to deliver the flow of air to the seal-forming structure 3100 at the entrance of the patient's airways. In the examples shown in FIGS. 5A-C, each tube 3350 lies in use on a path extending from the plenum chamber 3200 across the patient's cheek region and superior to the patient's ear to the elbow 3610. For example, a portion of each tube 3350 proximate the plenum chamber 3200 may overlie a maxilla region of the patient's head in use. Another portion of each tube 3350 may overlie a region of the patient's head superior to an otobasion superior of the patient's head. Each of the tubes 3350 may also lie over the patient's sphenoid bone and/or temporal bone and either or both of the patient's frontal bone and parietal bone. The elbow 3610 may be located in use over the patient's parietal bone, over the frontal bone and/or over the junction therebetween (e.g. the coronal suture).

As described above, in some examples of the present technology the patient interface 3000 comprises a seal-forming structure 3100 in the form of a cradle cushion which lies generally under the nose and seals to an inferior periphery of the nose (e.g. an under-the-nose cushion). The positioning and stabilising structure 3300, including the tubes 3350 may be structured and arranged to pull the seal-forming structure 3100 into the patient's face under the nose with a sealing force in a posterior and superior direction (e.g. a posterosuperior direction). A sealing force with a posterosuperior direction may cause the seal-forming structure 3100 to form a good seal to both the inferior periphery of the patient's nose and anterior-facing surfaces of the patient's face, for example on either side of the patient's nose and the patient's lip superior.

### 5.3.3 Cushion Module Connection to Frame

As has been described, the patient interface 3000 may comprise a frame 3360. When assembled, the cushion module 6100 is connected to the frame 3360 to hold the cushion module 6100 in position so that the seal-forming structure 3100 may be held in sealing contact with the patient's face. In addition, the connection between the frame 3360 and the cushion module 6100 may allow the flow of breathable gas through the frame 3360 to the cushion module 6100 (for example, in the case of the forms shown in FIGS. 7 and 9) or from a volume inside the frame 3360 to the cushion module 6100 (for example, in the case of the form shown in FIGS. 10A-B). For example, when the cushion module 6100 and frame 3360 are connected together, openings in each component may be aligned to permit gas to flow between them. In some forms, when the cushion module 6100 and frame 3360 are connected together, the cushion clip 6300 of the cushion module 6100 may form a loop around the opening 3364 in the frame body 3362.

When disassembled, the cushion module 6100 may be able to be detached from the frame 3360. The cushion module 6100 may be able to be repeatedly and readily detached and re-attached to the frame 3360 so that the cushion module 6100, or parts thereof (for example the cushion 6200) may be able to be cleaned and/or replaced. Again, the use of the term “readily” in this context is intended to refer to the removal of the cushion module 6100 from the frame 3360 being something that a user (e.g. a patient, carer or clinician) is able to perform without undue force.

The ability to remove the cushion module 6100 from the frame 3360 may be advantageous in enabling recycling of components of the patient interface 3000 when the patient interface 3000, or parts thereof, are disposed of. Since different components may be formed from different materials, and different materials may require a separate recycling process, the ability to separate the parts into sub-parts formed of like materials enables more ready recycling.

In certain forms of the technology, for example the forms of technology shown in FIGS. 6-7, the cushion module 6100 may be connected with the frame 3360. For example, a non-patient-facing side of the cushion module 6100 may be connected to a patient-facing side of the frame 3360. In some forms, a non-patient-facing side of the second cushion clip portion 6320, for example a non-patient-facing side of wall portion 6324, may be connected to a patient-facing side of the frame 3360.

The frame body 3362 may comprise one or more protrusions extending outwardly from the patient-facing side of the frame body 3362 to facilitate connection of the frame 3360 to the cushion module 6100.

In some forms, the nature of the connection may be a press-fit connection. For example, one of the cushion module 6100 and the frame 3360 may comprise a protrusion and the other of the cushion module 6100 and the frame 3360 may comprise a recess configured to receive the protrusion. In the form shown in FIG. 6C, the wall portion 6324 of the second cushion clip portion 6320 comprises a cylindrical protruding flange 6327 around the plenum chamber inlet port 3205 extending outwardly from the wall portion in the anterior direction (from the perspective when the patient interface 3000 is being worn). This protruding flange 6327 is configured to be inserted into a corresponding recess on the patient-facing side of the frame 3360. In some forms, both the cushion module 6100 and the frame 3360 may comprise a protrusion and the two protrusions are configured to press-fit together. For example, in the form shown in FIG. 6C, the patient-facing side of the frame 3360 may comprise a cylindrical protruding flange extending posteriorly towards the patient, and the two flanges may be configured to press-fit together in sealing engagement in order to connect the cushion module 6100 and frame 3360 without permitting leak of the flow of breathable gas through the connection.

In other forms, the nature of the connection may be a snap-fit or other interlock. For example, in a form similar to that shown in FIG. 6C, the protruding flange 6327 may be configured to snap-fit to, or interlock with, the frame 6327, instead of a press-fit connection.

### 5.3.3.1 Lateral Interlocks

In certain forms of the technology, for example as shown in FIGS. 9 and 10, the patient interface 3000 may comprise a plurality of (for example, two) interlocks to connect the frame 3360 and the cushion module 6100. Each interlock may comprise sets of interlocking portions, which in some forms may comprise a protrusion and a recess that are configured to mate. In other forms, the sets of interlocking portions may comprise two protrusions that are configured to mate. In the following description of certain forms of the technology, the interlocks will be described with one component comprising a protrusion and another component comprising a recess. It should be appreciated that there are also provided forms of the technology in which the protrusion and recess are swapped between components, even if that configuration is not specifically described.

In the examples of FIGS. 9 and 10, the cushion module 6100 comprises first and second cushion module interlock portions 6110 on each lateral side of the non-patient-facing side (e.g. an anterior side) of the cushion module 6100. In certain forms, the interlock portions 6110 may comprise recesses in the cushion clip 6300 while in other forms the interlock portions 6110 may comprise recesses in the cushion 6200. In addition, the frame 3360 comprises first and second frame interlock portions 3366 on each lateral side of the patient-facing side (e.g. a posterior side) of the frame body 3362. Referring to FIG. 9D, it will be apparent how, when the cushion module 6100 is brought into connection with the frame 3360, the interlocks hold the frame 3360 in position relative to the cushion module 6100.

In the exemplary forms of FIGS. 9 and 10, the first and second frame interlock portions 3366 extend outwardly from a patient-facing side of the frame 3360 in a direction generally towards the mid-sagittal plane of the patient (when the patient interface 3000 is worn by the patient), for example in a direction that is substantially parallel to a frontal plane of the patient during use. Put another way, the frame interlock portions 3366, which may be protrusions, may extend generally towards each other and, in some forms may be substantially parallel. It will be appreciated that this feature may be possible due to the concave shape of the patient-facing side of the frame 3360 in the lateral direction. The first and second cushion module interlock portions 6110 may be recesses shaped to accommodate the protruding first and second frame interlock portions 3366. That is, the first and second cushion module interlock portions 6110 may be recesses extending in a direction generally towards the mid-sagittal plane of the patient (when the patient interface 3000 is worn by the patient), for example in a direction that is substantially parallel to a frontal plane of the patient during use.

In the forms of FIGS. 9 and 10, in order for the frame 3360 and cushion module 6100 to be able to be assembled and disassembled, the frame 3360 may be configured to flex to alter a distance between the first and second frame interlock portions 3366. The bendable nature of the frame 3360 has already been described and is referred to again. A user is able to apply a bending force to the frame 3360, for example by using the mechanical advantage of one or more of the rigidiser arms 3340, in order to separate the first and second frame interlock portions 3366 by a sufficient amount to enable disengagement from, or engagement with, the first and second cushion module interlock portions 6110.

In some such forms of the technology, the cushion clip 6300 may be configured to be more rigid than the frame 3360. It will be appreciated that this relatively greater rigidity may be achieved through a combination of the shape of these respective components and/or the materials used to make them. For example, the cushion clip 6300 may be formed from a stiffer material than the frame 3360. Additionally, or alternatively, the cushion clip 6300 may have a greater thickness than the frame 3360.

### 5.3.4 Vent

It has already been described above that, in certain forms of the technology, the patient interface 3000 includes a vent 3400 constructed and arranged to allow for the washout of exhaled gases, e.g. carbon dioxide, from the plenum chamber 3200.

The vents 3400 described earlier in this specification are vents that are provided to the cushion module 6100, for example comprised as part of plenum chamber 3200. In other forms of the technology, the vent 3400 may be comprised as part of another part of patient interface 3000 or another part of the respiratory system with which patient interface 3000 operates in use. For example, the vent 3400 may be provided to the inlet conduit that supplies breathable gas to the plenum chamber 3200 through the plenum chamber supply port 3205. The vent 3400 may be provided to an elbow 4700 or other decoupling structure, e.g. swivel, configured to connect to the plenum chamber supply port 3205.

### 5.3.5 Decoupling Structure(s)

In certain forms the patient interface 3000 includes at least one decoupling structure, for example, a swivel or a ball and socket. The decoupling structure may, in some forms, take the form of an elbow, such as elbow 4700.

### 5.3.6 Connection Port

In certain forms, the patient interface comprises a connection port 3600, which allows for fluid connection to the air circuit 4170. The connection port 3600 may be a port on a downstream end of the inlet conduit or elbow 4700, with an upstream end connecting to the plenum chamber supply port 3205. In alternative forms, the connection port 3600 and plenum chamber inlet port 3205 may be provided by the same opening in the plenum chamber 3200.

### 5.3.7 Forehead Support

In many of the illustrated exemplary forms of the technology, the patient interface 3000 does not include a forehead support, i.e. there is no component that, in use, is configured to rest against the patient's forehead in order to assist positioning and stabilising the seal-forming structure 3100 on the patient's face. Some patients may find a patient interface 3000 without a forehead support less intrusive and/or claustrophobic to wear.

In other forms of the technology, the patient interface 3000 may include a forehead support 3700, for example of the type shown in FIG. 3.

### 5.3.8 Anti-Asphyxia Valve

In certain forms, the patient interface 3000 includes an anti-asphyxia valve. An anti-asphyxia valve may function to provide an air path with ambient for the patient to breathe in the event that the RPT device 4000 ceases supplying breathable gas to the patient. An anti-asphyxia valve may particularly be provided in the case of a full-face mask.

### 5.3.9 Ports

In one form of the present technology, a patient interface 3000 includes one or more ports that allow access to the volume within the plenum chamber 3200. In one form this allows a clinician to supply supplementary oxygen. In one form, this allows for the direct measurement of a property of gases within the plenum chamber 3200, such as the pressure.

### 5.4 RPT Device

An RPT device 4000 in accordance with one aspect of the present technology comprises mechanical, pneumatic, and/or electrical components. The RPT device 4000 may be configured to generate a flow of air for delivery to a patient's airways, such as to treat one or more of the respiratory conditions described elsewhere in the present document.

### 5.5 Air Circuit

An air circuit 4170 in accordance with an aspect of the present technology is a conduit or a tube constructed and arranged to allow, in use, a flow of air to travel between two components such as RPT device 4000 and the patient interface 3000 or 3800.

In particular, the air circuit 4170 may be in fluid connection with the outlet of the RPT device 4000 and the patient interface. The air circuit may be referred to as an air delivery tube. In some cases there may be separate limbs of the circuit for inhalation and exhalation. In other cases a single limb is used.

In some forms, the air circuit 4170 may comprise one or more heating elements configured to heat air in the air circuit, for example to maintain or raise the temperature of the air. The heating element may be in a form of a heated wire circuit, and may comprise one or more transducers, such as temperature sensors. In one form, the heated wire circuit may be helically wound around the axis of the air circuit 4170. The heating element may be in communication with a controller of the RPT device 4000. One example of an air circuit 4170 comprising a heated wire circuit is described in U.S. Pat. No. 8,733,349, which is incorporated herewithin in its entirety by reference.

### 5.6 Humidifier

In one form of the present technology there is provided a humidifier 5000 (e.g. as shown in FIG. 5A) to change the absolute humidity of air or gas for delivery to a patient relative to ambient air. Typically, the humidifier 5000 is used to increase the absolute humidity and increase the temperature of the flow of air (relative to ambient air) before delivery to the patient's airways.

The humidifier 5000 may comprise a humidifier reservoir 5110, a humidifier inlet 5002 to receive a flow of air, and a humidifier outlet 5004 to deliver a humidified flow of air. In some forms, as shown in FIG. 5A and FIG. 5B, an inlet and an outlet of the humidifier reservoir 5110 may be the humidifier inlet 5002 and the humidifier outlet 5004 respectively. The humidifier 5000 may further comprise a humidifier base 5006, which may be adapted to receive the humidifier reservoir 5110 and comprise a heating element 5240.

### 5.7 Glossary

For the purposes of the present technology disclosure, in certain forms of the present technology, one or more of the following definitions may apply. In other forms of the present technology, alternative definitions may apply.

### 5.7.1 General

Air: In certain forms of the present technology, air may be taken to mean atmospheric air, and in other forms of the present technology air may be taken to mean some other combination of breathable gases, e.g. oxygen enriched air.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Pressure: Force per unit area. Pressure may be expressed in a range of units, including cmH₂O, g-f/cm² and hectopascal. 1 cmH₂O is equal to 1 g-f/cm² and is approximately 0.98 hectopascal (1 hectopascal=100 Pa=100 N/m2=1 millibar 0.001 atm). In this specification, unless otherwise stated, pressure is given in units of cmH₂O.

Respiratory Pressure Therapy: The application of a supply of air to an entrance to the airways at a treatment pressure that is typically positive with respect to atmosphere.

Ventilator: A mechanical device that provides pressure support to a patient to perform some or all of the work of breathing.

### 5.7.1.1 Materials

Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45 as measured using ASTM D2240.

Polycarbonate: a thermoplastic polymer of Bisphenol-A Carbonate.

### 5.7.1.2 Mechanical Properties

Resilience: Ability of a material to absorb energy when deformed elastically and to release the energy upon unloading.

Resilient: Will release substantially all of the energy when unloaded. Includes e.g. certain silicones, and thermoplastic elastomers.

Hardness: The ability of a material per se to resist deformation (e.g. described by a Young's Modulus, or an indentation hardness scale measured on a standardised sample size).
‘Soft’ materials may include silicone or thermo-plastic elastomer (TPE), and may, e.g. readily deform under finger pressure.
‘Hard’ materials may include polycarbonate, polypropylene, steel or aluminium, and may not e.g. readily deform under finger pressure.

Stiffness (or rigidity) of a structure or component: The ability of the structure or component to resist deformation in response to an applied load. The load may be a force or a moment, e.g. compression, tension, bending or torsion. The structure or component may offer different resistances in different directions. The inverse of stiffness is flexibility.

Floppy structure or component: A structure or component that will change shape, e.g. bend, when caused to support its own weight, within a relatively short period of time such as 1 second.

Rigid structure or component: A structure or component that will not substantially change shape when subject to the loads typically encountered in use. An example of such a use may be setting up and maintaining a patient interface in sealing relationship with an entrance to a patient's airways, e.g. at a load of approximately 20 to 30 cmH2O pressure.

As an example, an I-beam may comprise a different bending stiffness (resistance to a bending load) in a first direction in comparison to a second, orthogonal direction. In another example, a structure or component may be floppy in a first direction and rigid in a second direction.

### 5.7.2 Patient Interface

Anti-asphyxia valve (AAV): The component or sub-assembly of a mask system that, by opening to atmosphere in a failsafe manner, reduces the risk of excessive CO2 rebreathing by a patient.

Elbow: An elbow is an example of a structure that directs an axis of flow of air travelling therethrough to change direction through an angle. In one form, the angle may be approximately 90 degrees. In another form, the angle may be more, or less than 90 degrees. The elbow may have an approximately circular cross-section. In another form the elbow may have an oval or a rectangular cross-section. In certain forms an elbow may be rotatable with respect to a mating component, e.g. about 360 degrees. In certain forms an elbow may be removable from a mating component, e.g. via a snap connection. In certain forms, an elbow may be assembled to a mating component via a one-time snap during manufacture, but not removable by a patient.

Frame: Frame will be taken to mean a mask structure that bears the load of tension between two or more points of connection with a headgear. A mask frame may be a non-airtight load bearing structure in the mask. However, some forms of mask frame may also be air-tight.

Headgear: Headgear will be taken to mean a form of positioning and stabilizing structure designed for use on a head. For example the headgear may comprise a collection of one or more struts, ties and stiffeners configured to locate and retain a patient interface in position on a patient's face for delivery of respiratory therapy. Some ties are formed of a soft, flexible, elastic material such as a laminated composite of foam and fabric.

Membrane: Membrane will be taken to mean a typically thin element that has, preferably, substantially no resistance to bending, but has resistance to being stretched.

Plenum chamber: a mask plenum chamber will be taken to mean a portion of a patient interface having walls at least partially enclosing a volume of space, the volume having air therein pressurised above atmospheric pressure in use. A shell may form part of the walls of a mask plenum chamber.

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Shell: A shell will be taken to mean a curved, relatively thin structure having bending, tensile and compressive stiffness. For example, a curved structural wall of a mask may be a shell. In some forms, a shell may be faceted. In some forms a shell may be airtight. In some forms a shell may not be airtight.

Stiffener: A stiffener will be taken to mean a structural component designed to increase the bending resistance of another component in at least one direction.

Strut: A strut will be taken to be a structural component designed to increase the compression resistance of another component in at least one direction.

Swivel (noun): A subassembly of components configured to rotate about a common axis, preferably independently, preferably under low torque. In one form, the swivel may be constructed to rotate through an angle of at least 360 degrees. In another form, the swivel may be constructed to rotate through an angle less than 360 degrees. When used in the context of an air delivery conduit, the sub-assembly of components preferably comprises a matched pair of cylindrical conduits. There may be little or no leak flow of air from the swivel in use.

Tie (noun): A structure designed to resist tension.

Vent: (noun): A structure that allows a flow of air from an interior of the mask, or conduit, to ambient air for clinically effective washout of exhaled gases. For example, a clinically effective washout may involve a flow rate of about 10 litres per minute to about 100 litres per minute, depending on the mask design and treatment pressure.

### 5.8 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

1. A patient interface for use in delivering breathable gas to a patient, the patient interface comprising:
a cushion module; and
a frame,
wherein the cushion module comprises:
a plenum chamber pressurisable to a therapeutic pressure of at least 4 cmH₂O above ambient air pressure, said plenum chamber forming a plenum chamber inlet port configured to receive a flow of breathable gas at the therapeutic pressure for breathing by the patient; and
a seal-forming structure provided to the plenum chamber, wherein the seal-forming structure is configured to form a seal with a region of the patient's face surrounding an entrance to the patient's airways, said seal-forming structure having an opening therein such that in use the flow of breathable gas is delivered to the entrance to the patient's airways, the seal-forming structure being configured to maintain said therapeutic pressure in the plenum chamber throughout the patient's respiratory cycle in use;
a first cushion module interlock portion on a first lateral side of a non-patient-facing side of the cushion module;
a second cushion module interlock portion on a second lateral side of the non-patient-facing side of the cushion module,
wherein the frame comprises:
a frame body forming a frame opening allowing the flow of breathable gas to be delivered to the plenum chamber;
two or more headgear connectors for connecting with headgear, the headgear connectors being positioned on respective lateral sides of the frame body in use;
a first frame interlock portion on a first lateral side of a patient-facing side of the frame body;
a second frame interlock portion on a second lateral side of the patient-facing side of the frame body,
wherein the frame and the cushion module are configured to connect together via a first interlock between the first cushion module interlock portion and the first frame interlock portion and via a second interlock between the second cushion module interlock portion and the second frame interlock portion, and
wherein the frame is configured to flex to alter a distance between the first frame interlock portion and the second frame interlock portion, and wherein flexing the frame to increase the distance between the first frame interlock portion and the second frame interlock portion enables the cushion module to be disconnected from the frame.

2. A patient interface as claimed in claim 1, wherein the first cushion interlock portion comprises a first recess, the second cushion interlock portion comprises a second recess, the first frame interlock portion comprises a first protrusion, and the second frame interlock portion comprises a second protrusion.

3. A patient interface as claimed in claim 1, wherein the patient-facing side of the frame body is concave.

4. A patient interface as claimed in claim 2, wherein the first frame interlock portion and the second frame interlock portion extend outwardly from a patient-facing side of the frame in a direction substantially parallel to a frontal plane of the patient during use.

5. A patient interface as claimed in claim 1, wherein the cushion module comprises a cushion and a cushion clip, wherein the first cushion module interlock portion and the second cushion module interlock portion are provided on the cushion clip.

6. A patient interface as claimed in claim 5, wherein the cushion clip forms a loop around the plenum chamber inlet port.

7. A patient interface as claimed in claim 5, wherein, when the cushion module is connected to the frame, the cushion clip forms a loop around the frame opening.

8. A patient interface as claimed in claim 5, wherein the cushion clip is more rigid than the frame.

9. A patient interface as claimed in claim 1, wherein the plenum chamber substantially defines a volume of space and the plenum chamber comprises an anterior wall substantially forming an anterior boundary of the volume of space during use.

10. A patient interface as claimed in claim 1, wherein the plenum chamber and the frame together substantially define a volume of space and the frame forms an anterior boundary of the volume of space during use.

11. A patient interface as claimed in claim 1, wherein the plenum chamber inlet port is located in a central region of the plenum chamber.

12. A patient interface as claimed in claim 1, wherein the plenum chamber inlet port is a first plenum chamber inlet port located on a first lateral side of the plenum chamber and the plenum chamber further forms a second plenum chamber inlet port located on a second lateral side of the plenum chamber, wherein the second plenum chamber inlet port is configured to receive the flow of breathable gas.

13. A patient interface as claimed in claim 1, wherein the frame opening is positioned substantially centrally in the frame body.

14. A patient interface as claimed in claim 1, wherein the frame body comprises an elongate hollow body with openings at each lateral end, wherein the openings are configured to receive the flow of breathable gas.

15. A patient interface as claimed in claim 1, wherein the frame body is configured to bend relatively easily when a force is applied to the lateral edges of the frame body in order to push the lateral edges of the frame body towards or away from the patient's face in use and the frame body is configured to be difficult to bend when a force is applied to the superior and inferior edges of the frame body in order to push the superior and inferior edges of the frame body towards or away from the patient's face in use.

16. A patient interface as claimed in claim 1, wherein the patient interface further comprises a rigidiser arm extending laterally outwardly from each distal end of the frame body.

17. A patient interface as claimed in claim 1, wherein the frame body comprises a collar surrounding the frame opening.

18. A patient interface as claimed in claim 1, wherein the patient interface further comprises a vent configured to allow a continuous flow of gases exhaled by the patient from an interior of the plenum chamber to ambient, said vent being configured to maintain the therapeutic pressure in the plenum chamber in use.

19. A patient interface as claimed in claim 1, wherein the patient interface further comprises headgear for positioning and stabilising the patient interface in a therapeutically effective position on the patient's head in use.
